(19) United States
(12) Patent Application Publication
(10) Pub. No.: US 2016/0289678 A1
(43) Pub. Date: Oct. 6, 2016

(54) USE OF MICRORNA 146-A IN THE DIAGNOSIS, TREATMENT AND PREVENTION OF PICORNAVIRUS INFECTION AND MICRORNA 146-A ANTAGONISTS

(71) Applicants: DCB-USA LLC, Wilmington, DE (US); NATIONAL TAIWAN UNIVERSITY, TAIPEI (TW)

(72) Inventors: Sung-Liang Yu, Taipei City (TW); Bing-Ching Ho, Taipei City (TW); Pan-Chyr Yang, Taipei City (TW)

(21) Appl. No.: 15/038,124

(22) PCT Filed: Nov. 24, 2014
(86) PCT No.: PCT/US14/67075

§ 371 Date: May 20, 2016

Related U.S. Application Data
(60) Provisional application No. 61/907,645, filed on Nov. 22, 2013.

(51) Int. Cl. C12N 15/113; G01N 33/569; C12Q 1/68

(52) U.S. Cl. CPC C12N 15/113; C12Q 1/6883; G01N 33/56983; C12N 2310/113; C12Q 2600/178; C12Q 2600/136; C12N 2320/30; G01N 2333/085; G01N 2500/10; C12N 2310/3231

(57) ABSTRACT

The present invention found that host miRNAs might be involved in Picornavirus pathogenesis through suppression of type I IFNs induction and could act as candidates for developing antiviral therapy. Thus, the invention suggests enterovirus-induced miR-146a facilitates viral pathogenesis by suppressing IFN production and provide a clue to develop the preventive and therapeutic strategies for enterovirus infections.

### FIELD OF THE INVENTION

The present invention relates to the field of microRNA (miRNA), in particular miR-146a and its antagonists for the diagnosis, prevention and/or therapy of Picornavirus infection,

### BACKGROUND OF THE INVENTION

Picornavirus is a group of small, non-enveloped viruses containing positive-strand RNAs coated by icosahedral protein shells. It causes a wide range of illnesses in both humans and animals, e.g., aseptic meningitis, encephalitis, the common cold, hand-foot-and-mouth disease, conjunctivitis, herpangina, and hepatitis. No medications are currently available for treating picornavirus infections. Picornavirus includes, but are not limited to, enterovirus (e.g., human enterovirus A, B, C, or D, poliovirus, and coxsackievirus), Rhinovirus (e.g., human rhinovirus A, B, or C), Hepatovirus (also known as Heparnavirus, such as Hepatitis A virus), Cardiovirus (e.g., Encephalomyocarditis virus), Aphthovirus (e.g., Foot-and-mouth disease virus).

Enteroviruses belong to the family Picornaviridae. They include about 70 human serotypes, e.g., polioviruses, coxsackieviruses A (COX A1-24), coxsackieviruses B (COX B1-6), echoviruses 1-31, enteroviruses (EV68-71), and enterovirus 72 (hepatitis A). Genomic sequences among various enteroviruses are well conserved. The virion of an enterovirus consists of a simple virus capsid and a single strand of RNA. Enteroviruses primarily enter the body through the alimentary canal. They replicate in the cell lining of the alimentary canal before spreading throughout the body via the blood circulation. Clinical syndromes of enteroviral infections are generally mild. Occasionally, enteroviruses cause serious diseases such as paralytic poliomyelitis, meningitis, or myocarditis.

Enterovirus 71 (EV71), a positive-stranded RNA genome encapsulated in nonenveloped icosahedral virion, is a member of the enterovirus genus of the Picornaviridae family EV71 possessed extensive tissues tropism that could infect center neuronal system, heart, lung, skeletal muscle, and intestine and its infection caused typical hand-foot-and-mouth disease, aseptic meningitis, encephalomyelitis, pulmonary edema, heart failure, poliomyelitis-like paralysis or even neurologic and psychiatric effects. EV71 was first identified in California in 1969. Several outbreaks were occurred in Bulgaria in 1975, Hungary in 1978, Malaysia in 1997, Taiwan in 2000 and China in 2010 and 2011 and resulted in dozens of deaths. EV71 has become a newly emerging life-threatening pathogen, particularly in the Asia-Pacific region recently. Unfortunately, there is no effective therapy or vaccine for EV71 infection (Solomon, T. et al. Virology, epidemiology. pathogenesis, and control of enterovirus 71. The Lancet infectious diseases 10, 778-790 (2010)).

Generally, virus infections can elicit interferons (IFNs) production due to the stimulation of single strand RNA, double strand RNA or hypomethylated CpG-DNA occurred in viral replication. Virus-associated molecules are recognized by host pattern-recognition receptors and activate the endosomal toll-like receptor (TLR) signallings to produce type I IFNs. The resulting IFNs and proinflammatory cytokines activate host adaptive immunity leading to completing host antiviral machinery. Type I IFNs can promote memory T cells proliferation, induce IFNγsecretion, and activate dendritic cells and natural killer cells. Thus, virus-infected individuals could establish antiviral machinery, possess abilities to inhibit viral replication and clean virus-infected cells. Intriguingly, EV71 could not effectively stimulate infected-hosts to produce type I IFNs in human being and in animal models. However, type I IFNs treatment could improve and even cure the EV71 infections (Hung, H. C., et al. Synergistic inhibition of enterovirus 71 replication by interferon and rupintrivir. J infect Dis 203, 1784-1790 (2011);Yi, L., He, Y., Chen, Y. Kung, H. F. & He, M. L. Potent inhibition of human enterovirus 71 replication by type I interferon subtypes. Antivir Ther 16, 51-58 (2011)). These clues implied that the sequelae and mortality caused by EV71 might be eased if type IFNs production can be normally induced during infection.

U.S. Pat. No. 6,815,444 provides pyrazolopyrimidine compounds for use as a therapeutic agent to treat enteroviral infection. U.S. Pat. No. 7,482,006 relates to anti-viral therapeutics, particularly recombinant human anti-EV71 monoclonal antibodies and application of said antibodies in therapy, surgery and diagnosis of EV71 infection. U.S. Pat. No. 7,718,775 provides a monoclonal antibody capable of neutralizing EV71 infection. U.S. Pat. No. 8,313,750 provides a capsid protein VP1 from human enterovirus 71 (EV71), “MEL701-VP1, used as a vaccine against EV71 U.S. Pat. No. 7,858,770 relates to an siRNA (small interfering RNA) having antiviral activity against nonpolio enteroviruses, and a pharmaceutical composition comprising same as an active ingredient for preventing and treating diseases caused by nonpolio enterovirus infection. However, the above-mentioned prior references are of no relevance to microRNA.

MicroRNAs (miRNAs) are an abundant class of short endogenous RNAs that act as post-transcriptional regulators of gene expression by base-pairing with their target mRNAs. The mature miRNAs are processed sequentially from longer hairpin transcripts by the RNAse III ribonucleases Drosha. miRNAs are highlighted and known to govern a wide range of biological functions including cellular proliferation, differentiation and apoptosis by post-transcriptional regulation of target gene expression. It is one long-held belief that virus infections could alter host gene expression profiles including miRNAs and that might contribute to viral propagation and pathogenesis. A previous study showed that EV71 infection reshapes gene and miRNA expressions. EV71 upregulates miR-141 expression through induction of EGR1 whereby virus could suppress host eukaryotic initiation factor 4E resulting in shutdown of cap-dependent translation and augment of virus propagation (Ho, B. C., et Enterovirus-induced miR-141 contributes to shutoff of host protein translation by targeting the translation initiation factor eIF4E. Cell host & microbe 9, 58-69 (2011)). Therefore, miRNAs may serve as targets or antiviral therapy.

### SUMMARY OF THE INVENTION

The invention provides a single strand oligonucleotide, which has a length of 8-25 nucleobase units, wherein the oligonucleotide comprises a seed nucleobase sequence consisting of AGTTCTCA (SEQ ID NO: 1) counting from 3′ end of the oligonucleotide. In one embodiment, the oligonucleotide of the invention is typically single stranded. Preferably, the single stranded oligonucleotide according to the invention comprises a region of contiguous nucleobase sequence which is 100% complementary to the miR-146a. The single stranded oligonucleotide of the invention can be used as miR-146a antagonist. In some embodiments, the contiguous nucleotide sequence of the single stranded oligonucleotide is between 8-25 nucleotides in length, such as 12, 13, 14, 15, 16, 17, 18, 19, 20, 21, 22, 23, 24 or 25 nucleobase units, wherein at least 50% of the nucleobase units of the single stranded oligonucleotide comprises nucleotide analogues. Preferably, the single stranded oligonucleotide comprises nucleotide analogues, such as LNA, which form part of, or may form the entire contiguous nucleotide sequence.

The invention also provides a method for diagnosis of Picornavirus infection, comprising the steps of:
providing a sample of a subject supposed to suffer from Picornavirus infection;
measuring the expression or promoter activity of miR-146a, c-jun, c-fos, IRAK1 and/or TRAF6;
wherein an elevated level of miR-146a and an elevated level of c-jun and/or c-fos or a reduced level of IRAK1 and/or TRAF6 in comparison to a control sample indicates Picornavirus infection.

The invention also provides a method for screening of a pharmaceutically active compound for the treatment and/or the prevention of Picornavirus infection, comprising the steps of:
providing a cell infected with Picornavirus;
contacting a candidate substance with the cell; and
measuring the expression or promoter activity of miR-146a, c-jun, c-fos, IRAK1 and/or TRAF6 in the cell;
   wherein a reduced level of miR-146a and a reduced level of c-jun and/or c-fos or an elevated level of IRAK1 and/or TRAF6 in comparison to a control sample indicates a pharmaceutically active compound.

The invention also provides a method for neutralizing Picornavirus, comprising contacting a miR-146a antagnoist with the Enterovirus virus, wherein the miR-146a antagonist is the single strand oligonucleotide as described herein. Also provided is a method for treating and/or preventing Picornavirus infection, comprising administering an effective amount of miR-146a is antagnoist to a subject, wherein the miR-146a antagonist is the single strand oligonucleotide as described herein. In some embodiments, the Picornavirus is Enterovirus. Preferably, the Enterovirus is Enterovirus A, Enterovirus B or Enterovirus C, more preferably, the Enterovirus is Enterovirus 71.

### BRIEF DESCRIPTION OF THE DRAWING

FIG. 1 shows IRAK1 and TRAF6 are the targets of EV71-induced miR-146a. a, miR-146a was induced in EV71 infection quantified by real-time RT-PCR. MI: mock infection; h.p.i., hours post-infection. b, EV71 infection suppressed IRAK1 and TRAF6 expressions in protein level but not in mRNA. c, Predicted miR-146a binding sites within Homo IRAK1 and TRAF6 3′UTRs. Two and three potential miR-146a binding sites located within IRAK1 or TRAF6 3′UTR, respectively (the first nucleotide following the stop codon was designated as +1). d, The effect of miR-1.46a on the luciferase reporter vectors harboring wild-type or mutant 3′UTRs. miR-146a significantly suppressed luciferase activities of vectors with wild-type 3′UTR but eliminated in vectors with mutant 3′UTRs (n=4). e, EV71 suppressed the expressions of V5-IRAK1 and V5-TRAF6 with wild-type 3′UTRs but not mutant ones. f, The effect of miR-146a on endogenous IRAK1 and TRAF6. MT: mock transfection; NC: negative control. All data present mean±s.d.

FIG. 2 shows regulation of miR-146a and the effect of EV71-induced miR-146a on interferon production. a, Schematic organization of miR-146a. AP1 (c-jun/c-fos) binding sites were predicted by intersection of TRANSFAC, PROMO and JASPAR software. b, AP1 was upregulated by EV71 infection. MI: mock infection; h.p.i.: hours post-infection. c, c-jun and c-fos activate miR-146a promoter. Transcriptional activities of miR 146a promoter were determined under indicated assay conditions. d, c-jun and c-fos enhance miR-146a expression. e, Determination of AP1 binding sites within miR-146a promoter. AP1 core sequence mutants were indicated in a. The transcriptional activities of miR146a mutant promoters were determined by luciferase activity assays in presence of c-jun and c-fos. f, EV71 activated miR-146a promoter harboring nature context but not mutant one. g, Silencing of AP1 decreased virus-induced miR-146a expression and restored IRAK1 and TRAF6. h, Suppression of IRAK1 and TRAF6 was restored by antagomiR-146a. i, Inhibition of IFNβ promoter activity was attenuated by antagomiR-146a. j, AntagomiR-146a eliminated the suppression of IFNβ expression in EV71 infection. All data present mean±s.d.

FIG. 3 shows induction of miR-146a is an universal phenomenon across cell types and enterovirus genus. a, miR-146a was induced in Caco-2 cells infected with EV71 MI: mock infection; h.p.i.: hours post-infection. b, EV71 infection suppressed IRAK1 and TRAF6 expressions in Caco-2 cells, c, AP1 (c-jun/c-fos) was induced in EV71 infection. mRNA and protein expression levels of c-jun and c-fos in EV71-infected Caco-2 cells were determined by real-time RT-PCR and Western blot, respectively. d, AntagomiR-146a restores EV71-induced suppression of TRAF6 and IRAK1 assayed by Western blot. e, miR-146a was induced in RD cells infected with PV3 or CVB3. f, Protein expression of IRAK'. and TRAF6 was suppressed in PV3 and CVB3 infections in RD cells. g, AP1 (c-jun/c-fos) was induced in PV3 and CVB3 infections in RD cells. All data present mean±s.d.

FIG. 4 shows mortality and suppression of IFN production are dramatically improved in mEV71-infected miR-146^{-/-} mice. a, Predicted miR-146a binding sites within Mus IRAK1 and TRAF6 3′UTRs. Three and two potential miR-146a binding sites located within Mus IRAK1 and TRAF6 3′UTRs, respectively (the first nucleotide following the stop codon was designated as +1). b, miR-146a^{-/-} mice displayed significant high survival probability. Each group was infected with indicated mEV71 PFUs and monitored for mortality daily. * p value presented comparison of 1*10⁸ PFU/miR-146a KO group and 1*10⁸ PFU/WT group; ** p value presented comparison of 2*10⁸ PFU/miR-146a KO group and 2*10⁸ PFU/WT group. c, Representative clinical signs and histological examinations in mEV71-infected mice, d, Virus propagations were restricted in miR-146a^{-/-} mice. Viral loads in indicated tissues were measured by plaque assays (n=4). e, miR-146a expressions were induced in mEV71-infected wild-type mice but not in mEV71-infected miR-146a^{-/-} mice (n=4). f, IRAK1 and TRAF6 expressions were suppressed in wild-type mice infected with mEV71 (n=4). g, IFNβ expressions were induced in miR-146a ^{-/-} mice infected with mEV71 IFNβ expressions were measured by real-time RT-PCR (n=4). All data present mean±s.d.

FIG. 5 shows LNA antagomiR-146a treatment improves survival and restores IFN production in EV71 mouse model. a, LNA antagomiR-146a treatment through intraperitoneal route improves survival probability. LNA antagomiR-146a was injected before (0 d.p.i.) or after (1 and 2 d.p.i.) virus infection and the mice survival was recorded daily. * p value presented comparison of 2*10 ⁸ PFU/anti-146a 1 d.p.i. group and 2*10⁸ PFU/anti-NC group; ** p value presented comparison of 2*10⁸ PFU/anti-146a 0 d.p.i. group and 2*10⁸ PFU/anti-NC group. b, The increase of miR-146a expression was neutralized in EV71-infected mice injected with LNA antagomiR-146a (n=4). c, Suppression of IRAK1 and TRAF6 was restored by LNA antagomiR-146a injection (n=4). d, IFNβ expression was induced in EV71-infected wild-type mice injected with LNA is antagomiR-146a by real-time RT-PCR. e, Anti-IFNα/β antibodies eliminated antagomiR-146a-mediated improved survival. LNA antagomiR-146a and anti-IFNα/β antibodies were sequentially injected before (0 d.p.i.) virus infection and the mice survival was recorded daily. p value presented comparison of 2*10⁸ PFU/anti-146a group and 2*10⁸ PFU/anti-NC group; ** p value presented comparison of 2*10⁸ PFU/anti-146a group and 2*10⁸ PFU/anti-146a/anti-IFNs group. f, Model for the regulatory role of miR-146a in enterovirus infection. AP1-mediated miR-146a induction represses IRAK1 and TRAF6 expression via imperfect base pairing between miR-146a and 3′UTRs of both genes. The reduction of IRAK1 and TRAF6, in turn, inhibits interferon production. EV71 escapes immune attacks by this new virus-host interaction and further causes viral pathogenesis including weight loss, paralysis and even death. Neutralization of miR-146a restores IRAK1 and TRAF6 expressions, restores IFN production and significantly improves survival. All data present mean±s.d.

FIG. 6 shows that IRAK1 and TRAF6 are the targets of mus miR-146a.

FIG. 7 shows the effects of designed antagomiR-146a_1 and antagomiR-146_2 on the luciferase reporter vectors harboring wild-type 3′UTRs in the presence of pSilencer-miR-146a.

### DETAILED DESCRIPTION OF THE INVENTION

The present invention found that host miRNAs might be involved in Picornavirus (preferably, Enterovirus and more preferably EV71) pathogenesis through suppression of type I IFNs induction and could act as candidates for developing antiviral therapy. Thus, the invention suggests enterovirus-induced miR-146a facilitates viral pathogenesis by suppressing IFN production and provide a clue to develop the preventive and therapeutic strategies for enterovirus infections.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference from what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

### Definitions

The terms “a” and “an” refer to one or more than one (i.e., at least one) of the grammatical object of the article.

As used herein, the term “or” in the claims refers to “and/or” unless explicitly indicated to refer to alternatives only or unless the alternatives are mutually exclusive.

The term “treat,” “treatment” or “treating” means reducing the frequency, extent, severity, and/or duration with which symptoms of infection of Picornavirus (preferably, Enterovirus and more preferably EV71) are experienced by a patient.

The term “prevent,” “prevention” or “preventing” means inhibition or the averting of symptoms associated with infection of Picornavirus (preferably, Enterovirus and more preferably EV71).

As used herein, the term “subject” refers to any recipient of a treatment, prevention or diagnosis using an agent or a treatment, prevention or diagnosis given for a similar purpose as described herein.

As used herein interchangeably, a “miR gene product,” “microRNA,” “miR,” or “miRNA” refers to the unprocessed or processed RNA transcript from a miR gene. As the miR gene products are not translated into protein, the term “miR gene products” does not include proteins. The unprocessed miR gene transcript is also called a “miR precursor,” and typically comprises an RNA transcript of about 70-100 nucleotides in length. The miR precursor can be processed by digestion with an RNAse (for example, Dicer, Argonaut, RNAse III (e.g., E. coli RNAse III)) into an active 21-23 nucleotide RNA molecule. This active 21-23 nucleotide RNA molecule is also called the “processed” miR gene transcript or “mature” miRNA.

The term “miR antagonist” means a single stranded oligonucleotide complementary to miR146a or a precursor or a modified oligonucleotide thereof. “Modified oligonucleotide” means an oligonucleotide having one or more modifications relative to a naturally occurring terminus, sugar, nucleobase, and/or internucleoside linkage. For example, “miR-146a antagonist” means a single stranded oligonucleotide complementary to miR146a or a modified oligonucleotide having nucleobase complementarity to miR-146a.

The term “LNA” refers to a bicyclic nucleotide analogue, known as “Locked Nucleic Acid”. It may refer to an LNA monomer, or, when used in the context of an “LNA oligonucleotide” refers to an oligonucleotide containing one or more such bicyclic nucleotide analogues.

The term “effective amount” means an amount of miRNAs effective to inhibit and/or treat and/or prevent infection caused by Picornavirus (preferably, Enterovirus and more preferably EV71). For example, the effective amount of the miRNAs may inhibit infection caused by Picornavirus (preferably, Enterovirus and more preferably EV71) and/or relieve to some extent one or more of the symptoms associated with the disorder caused by the infection.

### Isolated Single Strand Oligonucleotide of the Invention

in one aspect, the invention provides a single strand oligonucleotide or a nucleotide analogue thereof, which has a length of 8-25 nucleobase units, wherein the oligonucleotide comprises a seed nucleobase sequence consisting of AGTTCTCA (SEQ ID NO: 1) counting from 3′ end of the oligonucleotide.

The oligonucleotide of the invention is typically single stranded. It will therefore be understood that within the context of the invention the term oligonucleotide may be used interchangeably with the term single strand oligonucleotide. Moreover, in the context, the term “single stranded oligonucleotide” can be interchangeably used with the term “oligomer.”

In some embodiments, the contiguous nucleotide sequence of the single stranded oligonucleotide is between 8-25 nucleotides in length, such as 8, 9, 10, 11, 12, 13, 14, 15, 16, 17, 18, 19, 20, 21, 22, 23, 24 or 25 nucleobase units. In some embodiment, at least about 50%, about 60%, about 70%, about 80%, about 90%, about 92%, about 94%, about 95%, about 96%, about 97%, about 98% or about 99% of the nucleobase units of the single stranded oligonucleotide are complementary to the miR-146a sequence or a region thereof.

In some embodiments, the seed region counting from 3′ nucleobase of the single stranded oligonucleotide is complementary to the 5′ nucleotide of the seed region of the miR-146a, and the single stranded oligonucleotide comprises a contiguous nucleotide sequence which is fully complementary to the miR-146a seed sequence, and optionally between 1 and 17 further nucleotides, preferably 4 to 17 further nucleotides such as 4, 5, 6, 7, 8, 9, 10, 11, 12, 13, 14, 15, 16 or 17.

In one embodiment, the single strand oligonucleotide according to the invention comprises a region of contiguous nucleobase sequence which is 100% complementary to the miR-146a.

According to the invention, the miR-146a has the following sequence:

| | |
|---|---|
| UUGGGUACCUUAAGUCAAGAGU | (SEQ ID NO: 2) |

According to the invention, the single strand oligonucleotide of the invention can be used as miR-146a antagonist. Suitably, the single strand oligonucleotide is complementary (antimiR) to the miR-146a sequence or a region thereof, although it is considered that the single strand oligonucleotide may comprise one, two or few mismatches with the corresponding microRNA sequence or reverse complement thereof.

In some embodiments, the single strand oligonucleotide is an antimiR embodiment. The single strand oligonucleotide may be, in some embodiments, a linear molecule or is synthesized as a linear molecule. In some embodiments, the single strand oligonucleotide preferably does not comprise short regions of, for example, at least 3, 4 or 5 contiguous nucleotides, which are complementary to equivalent regions within the same single strand oligonucleotide (i.e. duplexes). In some embodiments, the single strand oligonucleotide may consist entirely of the contiguous nucleotide region. Thus, in some embodiments, the single stranded oligonucleotide is not substantially self-complementary.

When used herein, the term “nucleotide analogue” refers to a non-natural occurring nucleotide wherein, for example in one preferred embodiment, either the ribose unit is different from 2-deoxyribose and/or the nitrogenous base is different from A, C, T and G and/or the internucleoside phosphate linkage group is different. Suitable nucleotide analogues for use in the oligonucleotide of the invention are independently selected from the group consisting of: 2′-O-alkyl-RNA monomers, 2′-amino-DNA monomers, 2′-fluoro-DNA monomers, LNA monomers, arabino nucleic acid (ANA) monomers, 2′-fluoro-ANA monomers, HNA monomers, INA monomers.

2′-O-methoxyethyl-RNA, 2′-fluoro-DNA monomers and LNA are preferred and as such the oligonucleotide of the invention may comprise nucleotide analogues which are independently selected from these three types of analogue, or may comprise of only one type selected from the three types. In a most preferred embodiment the oligonucleotide comprises only LNA nucleotide analogues and nucleotides (RNA or DNA, most preferably DINA nucleotides).

Preferably, the single strand oligonucleotide comprises a nucleotide analogue, such as LNA, which form part of, or may form the entire contiguous nucleotide sequence.

In one embodiment the single stranded oligonucleotide, at least 60%, 65%, 70%, 75%, 80%, 85%, 90%, 95% or all of the nucleobase units of the contiguous nucleotide sequence are LNA nucleobase units. In one embodiment, all of the nucleobase units of the single strand oligonucleotide contiguous nucleotide sequence are LNA nucleobase units. In one embodiment the single stranded oligonucleotide, the contiguous nucleotide sequence comprises or consists of 4-17, preferably contiguous, nucleotide analogue units, such as LNA nucleobase units. Preferably, the single stranded oligonucleotide are selected from the group consisting of:

(SEQ ID NO: 3)

| | |
|---|---|
| 1. | 5′-UGAGAACUGAAUUCCAUGGGUU-3′ |
| | Designed antimiR-146a sequence (perfect match) |

(SEQ ID NO: 4)

5′- AACCCATGGAATTCAGTTCTCA-3′

(SEQ ID NO: 5)

| | |
|---|---|
| 2. | 5′-UGAGAAGUGAAUUCCAV(U to A, C, G)GGGUU-3′ |
| | Designed specific antimiR-146a sequence |

(SEQ ID NO: 6)

5′-AACCCB(T, G, C)TGGAATTCAGTTCTCA-3′

(SEQ ID NO: 7)

| | |
|---|---|
| 3. | 5′-UGAGAACUGAAUUCCAUH(G to A, T, C)GGUU-3′ |
| | Designed specific antimiR-146a sequence |

(SEQ ID NO: 8)

5′- AACCD(T, A, G)ATGGAATTCAGTTCTCA-3′

(SEQ ID NO: 9)

| | |
|---|---|
| 4. | 5′-UGAGAACUGAAUUCCAUGGH(G to A, T, C)UU-3′ |
| | Designed specific antimiR-146a sequence |

(SEQ ID NO: 10)

5′-AAD(T, A, G)CCATGGAATTCAGTTCTCA-3′

(SEQ ID NO: 11)

| | |
|---|---|
| 5. | 5′-UGAGAACUGAAUUCCAUGGGV(U to A, C, G)U-3′ |
| | Designed specific antimiR-146a sequence |

(SEQ ID NO: 12)

5′- AB(T, G, C)CCCATGGAATTCAGTTCTCA-3′

(SEQ ID NO: 13)

| | |
|---|---|
| 6. | 5′-UGAGAACUGAAUUCCAUGGGUV(U to A, C, G)-3′ |
| | Designed specific antimiR-146a sequence |

(SEQ ID NO: 13)

5′-B(T, G, C)ACCCATGGAATTCAGTTCTCA-3′

(SEQ ID NO: 14)

| | |
|---|---|
| 7. | 5′-UGAGAACUGAAUUCCAUGGGUUN(additional |
| | A, T, C, G)-3′ |
| | Designed specific antimiR-146a sequence |

(SEQ ID NO: 15)

5′-N(additional A, T, C, G)
AACCCATGGAATTCAGTTCTCA-3′

(SEQ ID NO: 16)

| | |
|---|---|
| 8. | 5′-UGAGAACUGAAUUCCAUGGGUUNN(additional |
| | A, T, C, G)-3′ |
| | Designed specific antimiR-146a sequence |

(SEQ ID NO: 17)

5′-NN(additional A, T, C, G)
AACCCATGGAATTCAGTTCTCA-3′

(SEQ ID NO: 18)

| | |
|---|---|
| 9. | 5′-UGAGAACUGAAUUCCAUGGGUUNNN(additional |
| | A, T, C,G)-3′ |
| | Designed specific antimiR-146a sequence |
| | (SEQ ID NO: 19) |
| | 5′-NNN(additional A, T, C, G) |
| | AACCCATGGAATTCAGTTCTCA-3′ |

(SEQ ID NO: 20)

| | |
|---|---|
| 10. | 5′-AACCCATGGAATTCAGTTCTCA-3′ |
| | (22 nucleotides; underline represented |
| | “seed region”) |

(SEQ ID NO: 21)

| | |
|---|---|
| 11. | 5′-ATGGAATTCAGTTCTCA-3′ |
| | (17 nucleotides) |

(SEQ ID NO: 22)

| | |
|---|---|
| 12. | 5′-ATTCAGTTCTCA-3′ |
| | (12 nucleotides) |

Whilst it is envisaged that other nucleotide analogues, such as 2′-MOE RNA or 2′-fluoro nucleotides may be useful in the antimiR oligomers according to the invention, in some embodiments the oligomers have a high proportion, such as at least 50%, LNA nucleotides. In one embodiment, at least 75%, such as 80% or 85% or 90% or 95% or all of the internucleoside linkages present between the nucleobase units of the contiguous nucleotide sequence are phosphorothioate internucleoside linkages. In one embodiment, said oligomer is conjugated with one or more non-nucleobase compounds. In one embodiment, the oligomer is constituted as a prodrug. In one aspect, the invention provides a pharmaceutical composition, comprising the single strand oligonucleotide of the invention. Preferably, the pharmaceutical composition further comprises a pharmaceutically acceptable carrier.

Methods for Diagnosis of Picornavirus Infection and Methods for Screening of a Pharmaceutically Active Compound for Treatment and/or Prevention of Picornavirus Infection

In one aspect, the present invention provides a method for diagnosis of Picornavirus infection, comprising the steps of:
(a) providing a sample of a subject supposed to suffer from Picornavirus infection;
(b) measuring the expression or promoter activity of miR-146a, c-jun, c-fos, IRAK1 and/or TRAF6;
wherein an elevated level of miR-146a and an elevated level of c-jun and/or c-fos or a reduced level of IRAK1 and/or TRAF6 in comparison to a control sample indicates Picornavirus infection.

In another aspect, the present invention relates to a method for screening of a pharmaceutically active compound for the treatment and/or the prevention of Picornavirus infection, comprising the steps of:
(a) providing a cell infected with Picornavirus;
(b) contacting a candidate substance with the cell; and
(c) measuring the expression or promoter activity of miR-146a, c-jun, c-fos, IRAK1 and/or TRAF6 in the cell;
wherein a reduced level of miR-146a and a reduced level of c-jun and/or c-fos or an elevated level of IRAK1 and/or TRAF6 in comparison to a control sample indicates a pharmaceutically active compound.

It is discovered herein that Picornavirus-induced mir-146a plays a critical role in Picornavirus infection. The invention surprisingly found that infection caused by Picornavirus (preferably, Enterovirus and more preferably EV71) induces miR-146a which targets to IRAK1 and TRAF6, two important proteins involved in the IFN production pathway, and suppresses their expressions. The infection caused by Picornavirus (preferably, Enterovirus and more preferably EV71) upregulates miR-146a expression which targets to IRAK1 and TRAF6 involved in TLR signalling and type I interferon production.

IRAK1 and TRAF6 are herein identified as the binding targets of miR-146a. Increasing miR-146a expression in the infection caused by Picornavirus (preferably, Enterovirus and more preferably EV71) suppresses expression of IRAK1 and TRAF6 and further reduces IFN production. AP1 is the most important transcriptional factor contributing Picornavirus-induced miR-146a upregulation (preferably, Enterovirus-induced miR-146a upregulation and more preferably EV71-induced miR-146a upregulation). It is found that virus-induced AP1 could upregulate miR-146a resulting in IRAK1 and TRAF6 suppression and c-jun and c-fos within the AP1 is the binding site as both c-jun and c-fos are significantly increased in the infection caused by Picornavirus (preferably, Enterovirus and more preferably EV71).

Accordingly, the expression of miR-146, c-jun, c-fos, IRAK1 and TRAF6 can be used as a marker to diagnose the infection caused by Picornavirus (preferably, Enterovirus and more preferably EV71) and screen a pharmaceutically active compound for the treatment and/or the prevention of the infection caused by Picornavirus (preferably, Enterovirus and more preferably EV71).

Method for Neutralizing Picornavirus and Method for Treating and/or Preventing Picornavirus Infection

In another aspect, the invention provides a method for neutralizing Picornavirus, comprising contacting a miR-146a antagnoist with the Enterovirus virus, wherein the miR-146a antagonist is the single strand oligonucleotide as described herein.

In a further aspect, the invention provides a method for treating and/or preventing is Picornavirus infection, comprising administering an effective amount of miR-146a antagnoist to a subject, wherein the miR-146a antagonist is the single strand oligonucleotide as described herein.

The present invention discovers that neutralization of Picornavirus-induced miR-146a rescues a subject suffering from Picornavirus infection from death via reproduction of type I interferon. Surprisingly, knockout of miR-146a or neutralization of virus-induced miR-146a by specific antagomiR, one kind of antimiR, restores the expression of IRAK1 and TRAF6 augments IFNβ production, inhibits viral propagation and improves survival in mouse models. The invention suggests that enterovirus-induced miR-146a facilitates viral pathogenesis by suppressing IFN production and provides a clue to develop the preventive and therapeutic strategies for enterovirus infections. Embodiments of the invention concern nucleic acids as miR-146a antagonists that perform the activities of inhibit endogenous miRNA-146a when introduced into cells.

Picornavirus includes, but are not limited to, enterovirus (e.g., human enterovirus A, B. C, or D, poliovirus, and coxsackievirus), Rhinovirus (e.g., human rhinovirus A, B, or C), Hepatovirus (also known as Heparnavirus, such as Hepatitis A virus), Cardiovirus (e.g., Encephalomyocarditis virus), Aphthovirus (e.g., Foot-and-mouth disease virus). The preferred Picornavirus is Enterovirus.

Enterovirus are a genus of positive-sense single-stranded RNA viruses associated with several human and mammalian diseases. The genera of Enterovirus are listed in the below table.

| | | |
|---|---|---|
| Enterovirus | Enterovirus A | 23 types: coxsackievirus A2 (CV-A2), CV-A3, CV-A4, CV-A5, CV- |
| | | A6, CV-A7, CV-A8, CV-A10, CV-A12, CV-A14, CV-A16, |
| | | enterovirus (EV) A71, EV-A76, EV-A89, EV-A90, EV-A91, EV-A92, |
| | | EV-114, EV-A119, SV19, SV43, SV46 & BA13; see also coxsackie A |
| | | virus |
| | Enterovirus B | 60 types: coxsackievirus B1 (CV-B1), CV-B2, CV-B3, CV-B4, CV- |
| | | B5 (incl. swine vesicular disease virus [SVDV]), CV-B6, CV-A9, |
| | | echovirus 1 (E-1; incl. E-8), E-2, E-3, E-4, E-5, E-6, E-7, E-9 (incl. |
| | | CV-A23), E-11, E-12, E-13, E-14, E-15, E-16, E-17, E-18, E-19, E- |
| | | 20, E-21, E-24, E-25, E-26, E-27, E-29, E-30, E-31, E-32, E-33, |
| | | enterovirus B69 (EV-B69), EV-B73, EV-B74, EV-B75, EV-B77, EV- |
| | | B78, EV-B79, EV-B80, EV-B81, EV-B82, EV-B83, EV-B84, EV- |
| | | B85, EV-B86, EV-B87, EV-B88, EV-B93, EV-B97, EV-B98, EV- |
| | | B100, EV-B101, EV-B106, EV-B107, EV-B110 & SA5; see also |
| | | coxsackie B virus and echovirus |
| | Enterovirus C | 23 types: poliovirus (PV) 1, PV-2, PV-3, coxsackievirus A1 (CV-A1), |
| | | CV-A11, CV-A13, CV-A17, CV-A19, CV-A20, CV-A21, CV-A22, |
| | | CV-A24, EV-C95, EV-C96, EV-C99, EV-C102, EV-C104, EV-C105, |
| | | EV-C109, EV-C113, EV-C116, EV-C117 & EV-118 |
| | Enterovirus D | 5 types: enterovirus D68 (EV-D68), EV-D70, EV-D94, EV-D111 & |
| | | EV-D120 |
| | Rhinovirus A | 77 types: human rhinoviris (HRV) A1, A2, A7, A8, A9, A10, A11, |
| | | A12, A13, A15, A16, A18, A19, A20, A21, A22, A23, A24, A25, |
| | | A28, A29, A30, A31, A32, A33, A34, A36, A38, A39, A40, A41, |
| | | A43, A44, A45, A46, A47, A49, A50, A51, A53, A54, A55, A56, |
| | | A57, A58, A59, A60, A61, A62, A63, A64, A65, A66, A67, A68, |
| | | A71, A73, A74, A75, A76, A77, A78, A80, A81, A82, A85, A88, |
| | | A89, A90, A94, A95, A96, A98, A100, A101, A102 and A103 |
| | Rhinovirus B | 25 types: human rhinovirus (HRV) B3, B4, B5, B6, B14, B17, B26, |
| | | B27, B35, B37, B42, B48, B52, B69, B70, B72, B79, B83, B84, B86, |
| | | B91, B92, B93, B97 and B99 |
| | Rhinovirus C | 51 types: human rhinovirus (HRV) C1-C51 |

Preferably, the Enterovirus is Enterovirus A, Enterovirus B or Enterovirus C. More preferably, the Enterovirus is Enterovirus 71.

In certain embodiments, it is desired to neutralize Picornavirus (preferably, Enterovirus and more preferably EV71) and/or treat and/or prevent the infection caused by Picornavirus (preferably, Enterovirus and more preferably EV71). The routes of administration will vary, naturally, with the location and nature of the site to be targeted, and include, e.g., intradermal, subcutaneous, regional, parenteral, intravenous, intramuscular, intranasal, systemic, and oral administration and formulation.

In some embodiments, the method for the delivery of a miRNA or an expression construct encoding such or combinations thereof is via systemic administration. However, the pharmaceutical compositions disclosed herein may also be administered orally, topically, parenterally, subcutaneously, directly, intratracheally, intravenously, intradermally, intramuscularly, or even intraperitoneally.

Parenteral administration, generally characterized by injection, either subcutaneously, intramuscularly or intravenously is also contemplated herein. If administered intravenously, suitable carriers include physiological saline or phosphate buffered saline (PBS), and solutions containing thickening and solubilizing agents, such as glucose, polyethylene glycol, and polypropylene glycol and mixtures thereof. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN.RTM. 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment. Injection of nucleic acids may be delivered by syringe or any other method used for injection of a solution, as long as the nucleic acid and any associated components can pass through the particular gauge of needle required for injection. A syringe system has also been described for use in gene therapy that permits multiple injections of predetermined quantities of a solution precisely at any depth. Injectables can be prepared in conventional forms, either as liquid solutions or suspensions, solid forms suitable for solution or suspension in liquid prior to injection, or as emulsions. Suitable excipients are, for example, water, saline, dextrose, glycerol, mannitol, 1,3-butanediol, Ringer's solution, an isotonic sodium chloride solution or ethanol.

In certain embodiments, oral pharmaceutical dosage forms are either solid, gel or liquid. The solid dosage forms are tablets, capsules, granules, and bulk powders. Types of oral tablets include compressed, chewable lozenges and tablets which can be enteric-coated, sugar-coated or film-coated. Capsules can be hard or soft gelatin capsules, while granules and powders can be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

In certain embodiments, pharmaceutical compositions are prepared for buccal administration. Certain of such pharmaceutical compositions are tablets or lozenges formulated in conventional manner.

Examples of binders for use in the compositions provided herein include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, sodium alginate, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

### EXAMPLES

### Example 1

### Targets of Virus-Induced miR-146a

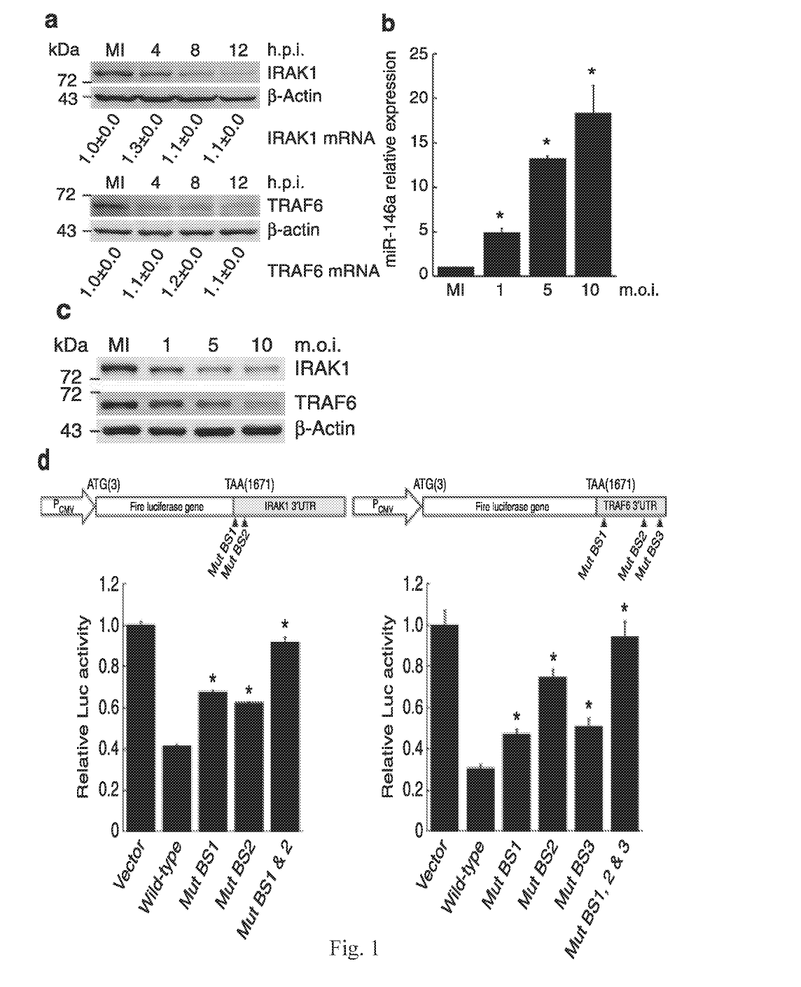

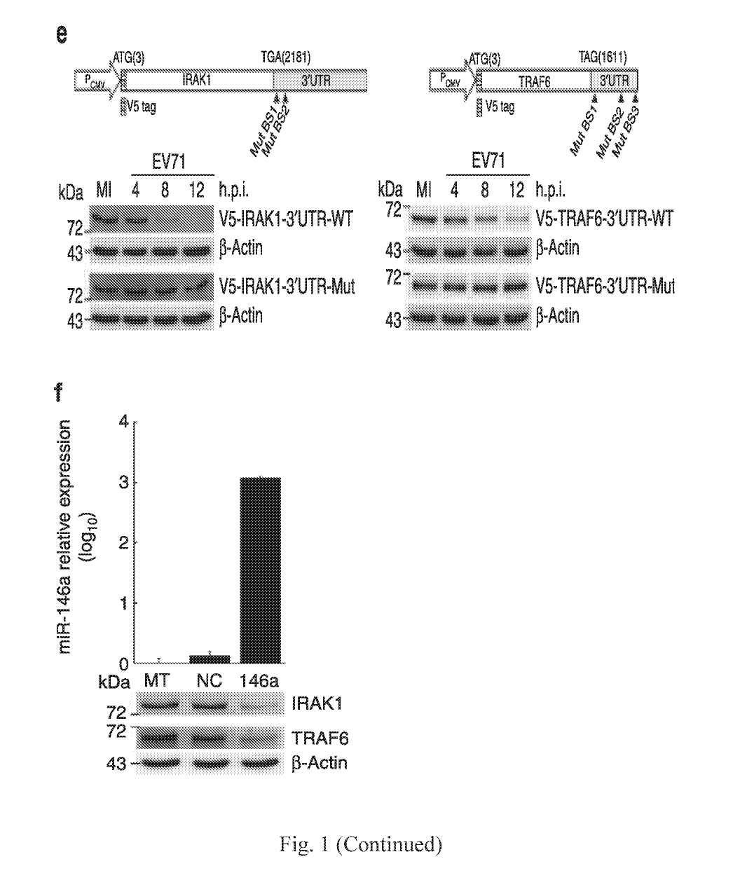

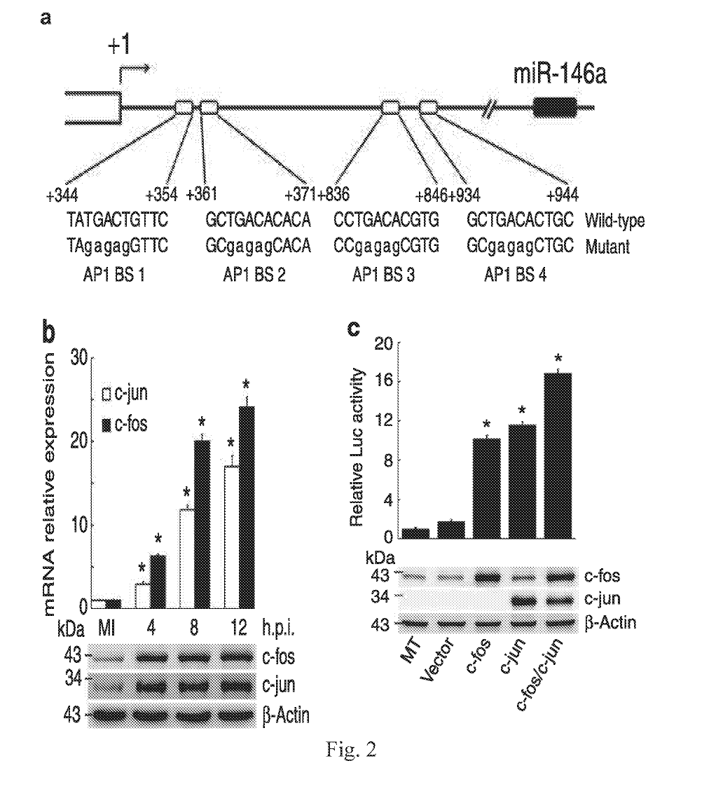

miR-146a, a EV71-induced microRNA, was selected for further investigation in this study due to its regulatory activity in TLR signalling and IFNs production (FIG. 1a). To determine whether IRAK1 and TRAF6 were affected by EV71 infection, we first detected IRAK1 and TRAF6 expressions at different post-infection time points. Both protein expressions were suppressed but mRNA expressions were not (FIG. 1b). Taken together, this result indicates miR-146a can be induced by EV71 infection and target to IRAK1 and TRAF6. However, the exact miR-146a binding sites of IRAK1 and TRAF6 are not thoroughly validated yet and the suppressive activity of individual binding sites is not evaluated actually. Hence, we further used the PicTar (http://pictar.org/) and RNA22 (http://cbcsrv.watson.ibm.com/rna22.html) to predict the potential miR-146a binding sites within the 3′UTR of IRAK1 and TRAF6 (FIG. 1c). Next, 3′UTRs of IRAK1 and TRAF6 with or without 4-base mutations in the predicted miR-146a binding sites were constructed into the miRNA reporter vectors. The reporter assays demonstrated that miR-146a suppresses luciferase activities of vectors harboring wild-type 3′UTR, less suppresses in vectors with single mutant binding site and almost losses its suppressive activity for vectors with combined mutant binding sites (FIG. 1d). We further elucidated whether the suppression of IRAK1 and TRAF6 in EV71 infection is specifically miR-146a-dependent, the V5-IRAK1-3′UTR-WT, V5-IRAK1-3′UTR-Mut (combined all mutant binding sites), V5-TRAF6-3′UTR-WT, and V5-TRAF6-3′UTR-Mut stable expression cells were established and infected with EV71 and the protein expression of V5-IRAK1 and V5-TRAF6 was measured at the indicated time points by Western blot. Both expressions of V5-IRAK1 and V5-TRAF6 with the wild-type 3′UTR were markedly suppressed. However, there was no obvious suppression on the expressions of V5-IRAK1 and V5-TRAF6 with the mutant 3′UTR. (FIG. 1e). FIG. 1f showed that the ectopic expression of miR-146a could directly suppress endogenous IRAK1 and TRAF6. We demonstrated that EV71 infection induces miR-146a which targets to IRAK1 and TRAF6, two important proteins involved in the IFN production pathway, and suppresses their expressions. However, the underlying mechanism by which miR-146a was upregulated in EV71 infection is still unclear and needs to be explored for deeply understanding EV71 pathogenesis.

### Example 2

### AP1 Upregulates miR-146a Expression

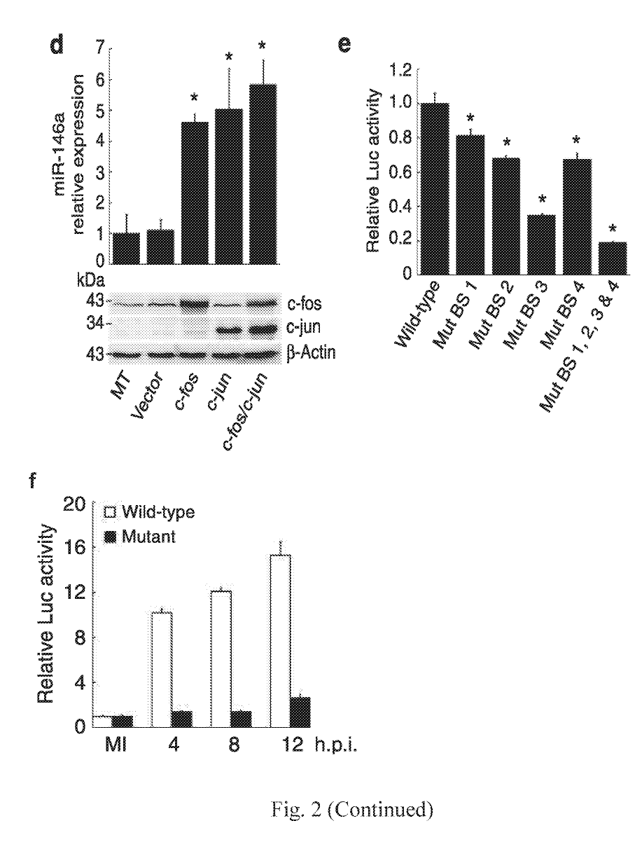

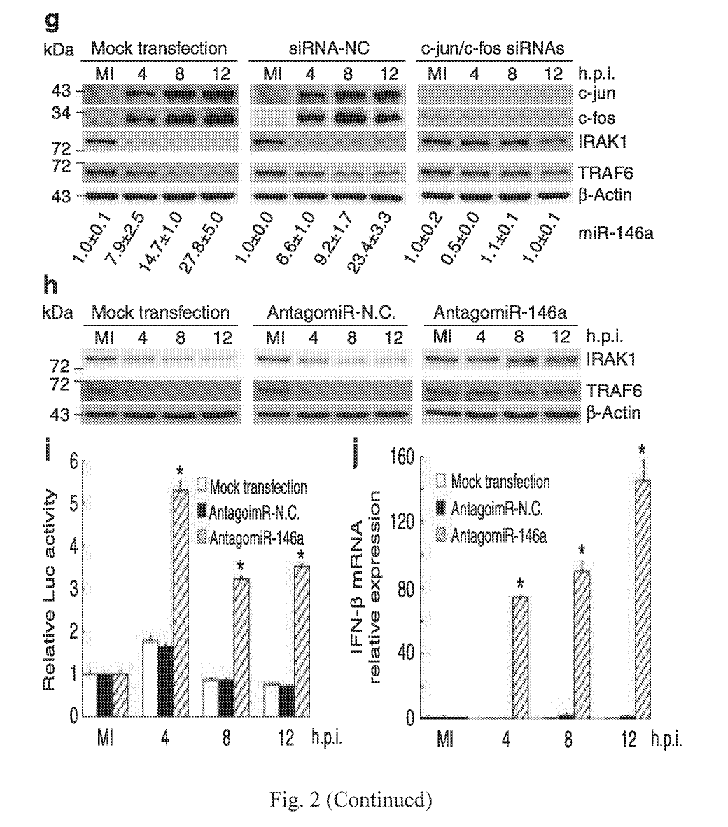

To determine which transcriptional factor(s) is responsible for the regulation of virus-induced miR-146a, we intersected the EV71-altered transcription factors assayed by microarrays and the potential transcription factors binding sites within miR-146a promoter region. AP1 (c-jun/c-fos) is the only one candidate identified in the intersection. Four potential AP1 binding sites were predicted within miR-146a promoter region (FIG. 2a). The expression of AP1 (c-jun/c-fos) was measured by real-time RT-PCR and Western blot and the results showed that both c-jun and c-fos were significantly increased in EV71 infection (FIG. 2b). The miR-146a promoter region was constructed into a luciferase reporter vector and assayed in presence of exogenously expressed c-jun and c-fos. Enforced expression of c-jun, c-fos or both transcription factors could enhance transcriptional activity of miR-146a promoter as well as endogenous miR-146a expression (FIGS. 2c and 2d).

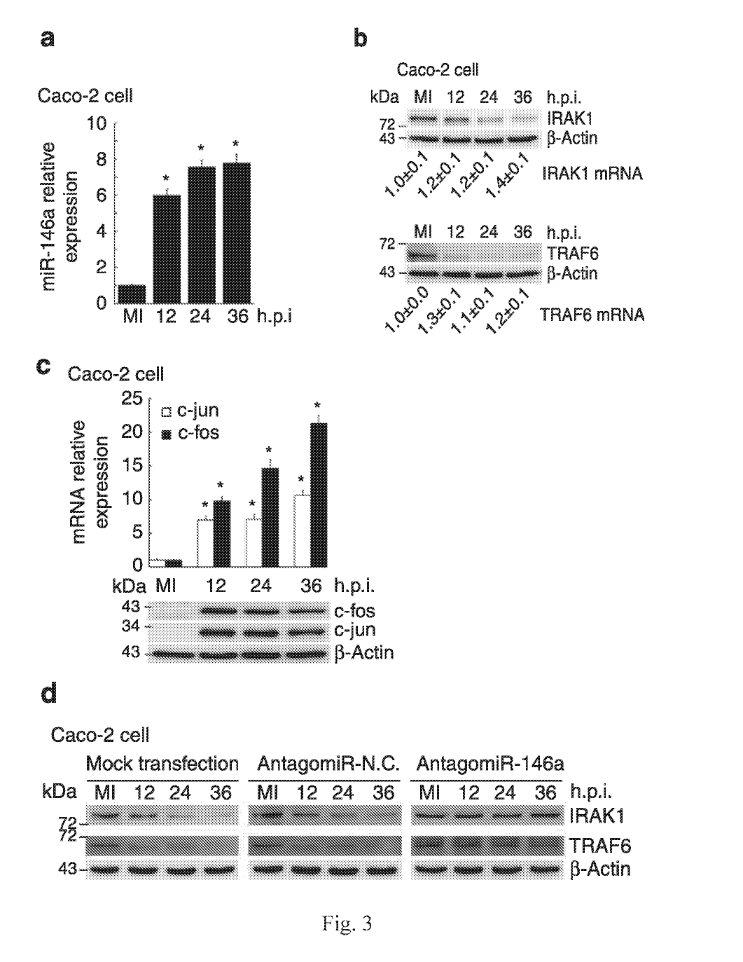

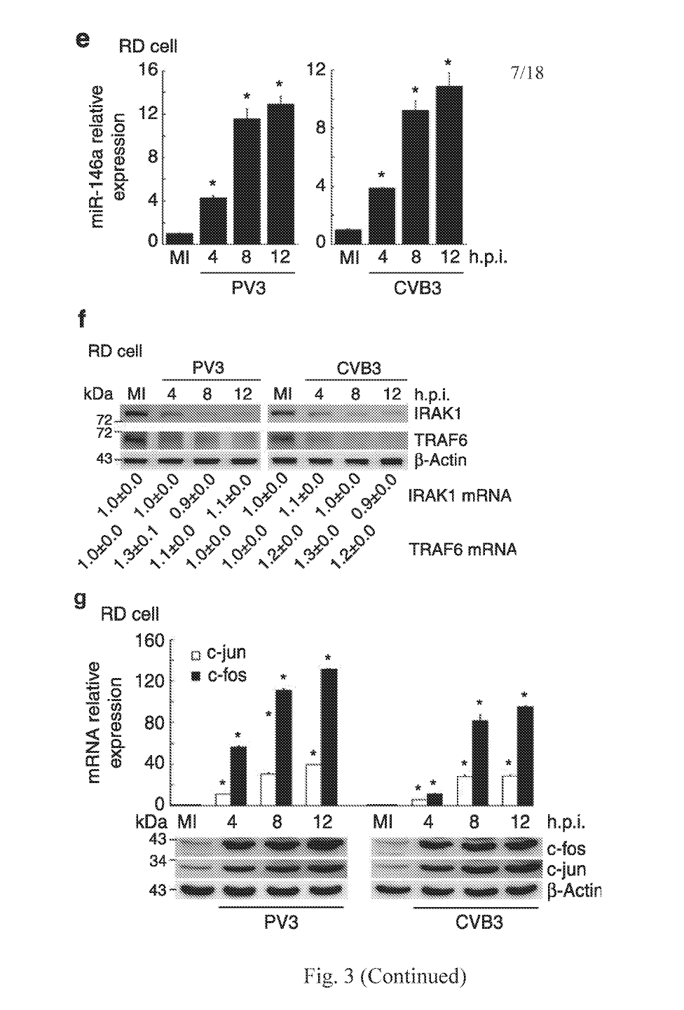

To further verify the direct activation activity of AP1 on miR-146a expression, we generated different mutation constructs for each potential AP1 binding site (BS) within miR-146a promoter region (FIG. 2a) and determined the importance of each predicted AP1 BS by reporter assays. The mutation of third potential AP1 BS impaired the transcriptional activity more severe than the other three potential AP1 BS under AP1 overexpression. However, all of four BS contributed to miR-146a upregulation because the luciferase activity of vector with four mutant AP1 BS was most suppressed (FIG. 2e). Moreover, wild-type and combined four AP1 BS mutant miR-146a promoter constructs were transfected into host cells followed by virus infection to evaluate the contribution of AP1 on miR-146a promoter activity. EV71 infection could induce transcriptional activity of wild-type miR-146a promoter but slight effect on mutant one (FIG. 2f). This data indicates that AP1 is the most important transcriptional factor contributing EV71-induced miR-146a upregulation. We suggest virus-induced AP1 could upregulate miR-146a resulting in IRAK1 and TRAF6 suppression. To address this issue, c-jun and c-fos siRNAs were introduced into host cells followed by virus infection and two target proteins, IRAK1 and TRAF6, and miR-146a were assayed by Western blot and real-time RT-PCR, respectively. miR-146a expression was inhibited and IRAK1 and TRAF6 were restored in host cells in presence of c-jun and c-fos siRNAs (FIG. 2g). JNK inhibitor (SP600125), acts as a reversible ATP-competitive inhibitor, could inhibit the activation of JNK pathway and further decreases c-jun and c-fos expression. Host cells were treated with JNK inhibitor (20 μM) before virus infection and assayed the expression levels of c-jun, c-fos, miR-146a, IRAK1 and TRAF6 at indicated hours post-infection (h.p.i.). AP1 expression was inhibited by JNK inhibitor accompanied with suppression of miR-146a and restoration of IRAK1 and TRAF6, as we expected. IRAK1 and TRAF6 are two key components in the signaling pathway of type I IFNs production. To explore whether recovery of IRAK1 and TRAF6 suppressed by virus infection could restore IFNs production, antagomiR-146a was used to neutralize virus-induced miR-146a. AntagomiR-146a was transfected into host cells followed by virus infection and IRAK1 and TRAF6 were recovered remarkably (FIG. 2h). Under this assay condition, IFNβ promoter activity was restored henceforth 4 h.p.i, compared with negative control or mock transfection (FIG. 2i). The IFNβ mRNA expression was increased dramatically in antagomiR-146a transfectants assayed by real-time RT-PCR (FIG. 2j). Neutralization of miR-146a rescued IRAK1 and TRAF6 expressions and in turn restored IFN production in EV71-infected cells. Additionally we explore whether this miR-146a signalling cascade also exits in another important EV71 primary targeting organ, intestine. Caco-2 cells, one kind of colon adenocarcinoma cells, were in place of RD cells to investigate the AP1-mediated miR-146a upregulation and IRAK1 and TRAF6 suppression upon EV71 infection. Caco-2 cells were infected with EV71 at multiplicity of infection of 10 and analyzed for the expression of c-jun, c-fos, miR-146a, IRAK1 and TRAF6 at indicated h.p.i. EV71 infection induced miR-146a expression and caused IRAK1 and TRAF6 suppression as we previously found in RD cells (FIGS. 3a and 3b). AP1 was upregulated in mRNA and protein levels started at 12 h.p.i. (FIG. 3c). Suppression of IRAK1 and TRAF6 could also be restored in the presence of antagomiR-146a compared with negative controls (FIG. 3d). These evidences implied AP1-mediated miR-146a induction and IRAK1 and TRAF6 suppression are universal regulations occurred in different EV71-infected cell types. We then investigated whether the upregulation of miR-146a is a common characteristic in enterovirus infections, the expression of miR-146a was measured in RD cells infected with two other enteroviruses, poliovirus 3 (PV3) and coxsackievirus B3 (CVB3). As shown in FIGS. 3e-3g, CVB3 and PV3 infections induced miR-146a increase and AP1 upregulation as well as IRAK1 and TRAF6 suppression. We have demonstrated the important role of miR-146a in type I IFN production in vitro and thus we further evaluated the role of miR-146a in virus infection in vivo especially focusing on IFNs production and pathogenesis.

### Example 3

### MiR-146a is Critical for EV71 Pathogenesis In Vivo

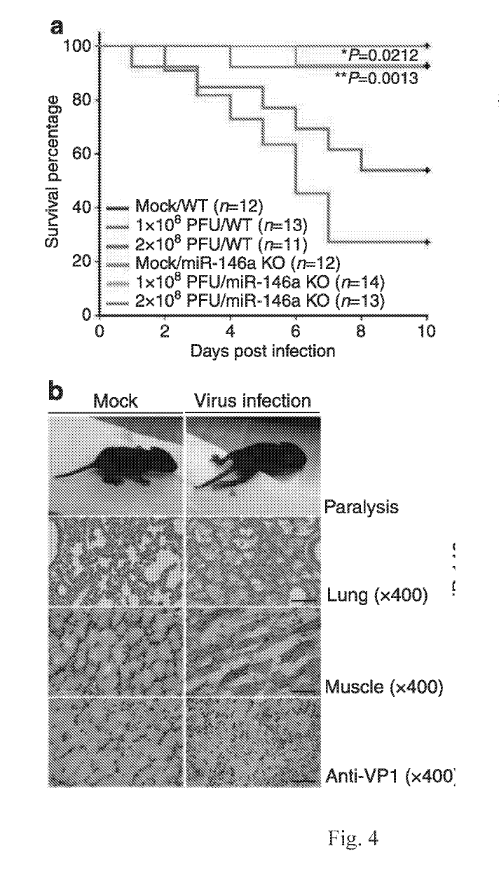

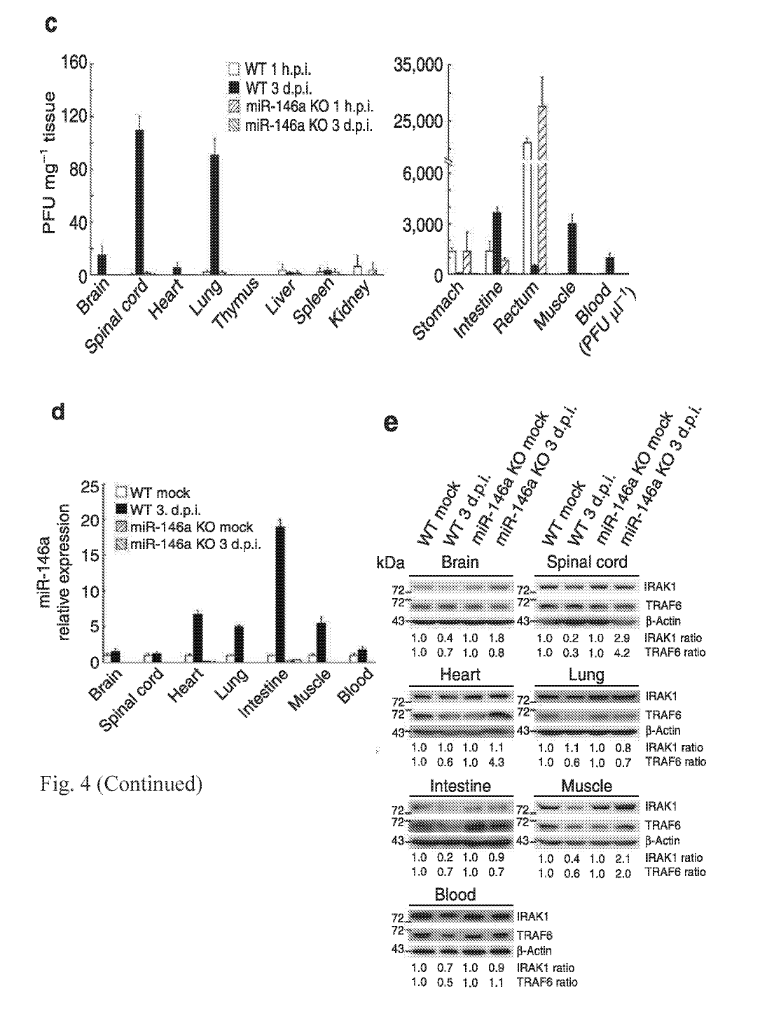

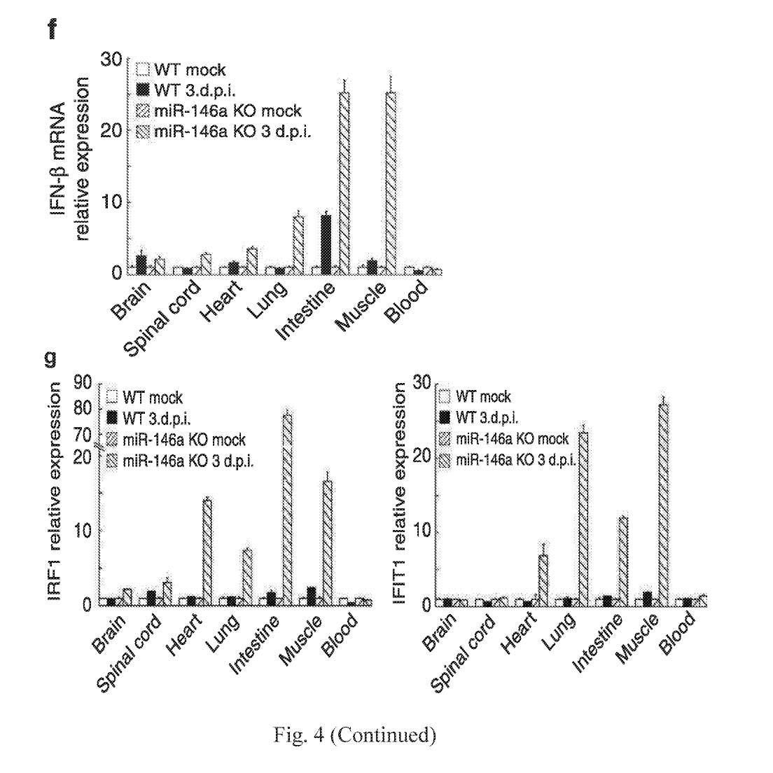

Because Mus miR-146a sequence is identical to Homo miR-146a sequence we speculate that Mus miR-146a might bind onto Mus IRAK1 and TRAF6 3′UTRs and suppress their expression. There are three potential binding sites within Mus IRAK1 3′UTR while two within Mus TRAF6 3′UTR (FIG. 4a). To characterize the role of miR-146a in EV71 infection in vivo, we first generated the mouse-adapted EV71 (named as mEV71 thereafter) for establishing EV71 infection mouse model due to low- or non-infectivity of human EV71 in mice¹. Different mEV71 plaque forming units (PFUs) were used to titrate the optimal mEV71 doses that will be used in the following in vivo mEV71 infection assays. The 10-day survivals of mice infected with 1×10⁸ and 2×10⁸ PFUs of mEV71 through naturally oral route were 60% and 20%, respectively and selected for further experiments. The miR-146a expressions of major organs in miR-146a knockout mice were measured by real-time RT-PCR. Although the miR-146a expressions of organs assayed were varied in wild-type mice but were not detectable in miR-146a^{-/-} mice.

Both wild-type and miR-146a^{-/-} mice were fed with two doses of mEV71 as indicated and recorded the clinical symptoms daily. The 10-day survivals of wild-type mice infected with 2×10⁸ and 1×10⁸ PFUs are 27% and 54%, respectively. Surprisingly, survivals of miR-146a infected with 2×10⁸ and 1×10⁸ PFUs significantly improved to 92% and 93% at 10 days post-infection (d.p.i.) (p=0.0013 and 0.0212, respectively) (FIG. 4b). It implied that knockout of miR-146a could improve the survival in EV71 infection. Next, we found that the mEV71-infected mice displayed rear-limb paralysis but mock-infected mice not (FIG. 4c, upper panel). Muscle tissues of virus-infected mice presented severe necrotizing myositis compared with mock-infected mice assayed by hematoxylin and eosin staining (FIG. 4c, middle panel). Previously Wang and his colleagues have reported that EV71 infection can induce necrotizing myositis. Hence, we further addressed whether necrotizing myositis was associated with virus infection and immunohistochemistry staining was performed using anti-EV71 specific antibody. EV71 was obviously detected in necrotizing myositis muscles (FIG. 4c, lower panel). Previous study demonstrated that high tissue viral load accompanied with severe illness signs and high mortality. Due to the dramatic difference of mortality between wild-type and miR-146a^{-/-} mice we measured EV71 titers in all major organs at 1 h.p.i. and 3 d.p.i. by plaque assays. It is reasonable that higher titers of EV71 were detected in the gastrointestinal tract (stomach, intestine and rectum) of both wild-type and miR-146a^{-/-} mice at 1. h.p.i. due to EV71 infection through oral administration. Three days later, the major EV71 susceptible organs (intestine, muscle, brain, spinal cord and lung) of wild-type mice have much higher viral loads compared with miR-146a^{-/-} mice (FIG. 4d).

Moreover, the presence of EV71 in blood of wild-type but not miR-146a^{-/-} mice at 3 d.p.i. indicated that viremia was only occurred in wild-type mice and miR-146a knockdown restricted EV71 spreading. These data implied a systemic EV71 infection occurred in wild-type mice but not miR-146a^{-/-} mice. Taken together, these findings reason why miR-146a^{-/-} mice are more resistant to EV71 infection and high viral loads could cause high mortality.

To further verify miR-146a-mediated signal transduction the expression levels of miR-146a, IRAK1, TRAF6 and IFNβ were assayed by real-time RT-PCR or Western blot. miR-146a expressions were much increased in heart, lung, intestine and muscle but less increased in brain, spinal cord and blood in wild-type mice upon EV71 infection (FIG. 4e). A reciprocal correlation was found between miR-146a expression and its target genes expressions, IRAK1 and TRAF6, that is, higher miR-146a expression was associated with lower expression of IRAK1 and TRAF6 in wild-type mice (FIG. 4f). Additionally, IFNβ production was also reciprocally correlated to miR-146a expression especially in heart, lung, intestine and muscle (FIG. 4g). In the case of miR-146a^{-/-} mouse, we could not detect any increased miR-146a expression in all assayed organs. Consistently, there is no obvious decreased expression of IRAK1 and TRAF6 or IFNβ inhibition compared with mock control group (FIGS. 4f-4g). Furthermore, the body weights of wild-type mice infected with 2×10⁸ PFUs were dramatically decreased compared with EV71-infected miR-146a^{-/-} mice group or mock groups. These data clearly indicated that miR-146a governs EV71 pathogenesis including viral propagation, IFNs blockage, clinical illness and even mortality.

### Example 4

### LNA AntagomiR-146a Provides a Potential Therapy Against Enterovirus

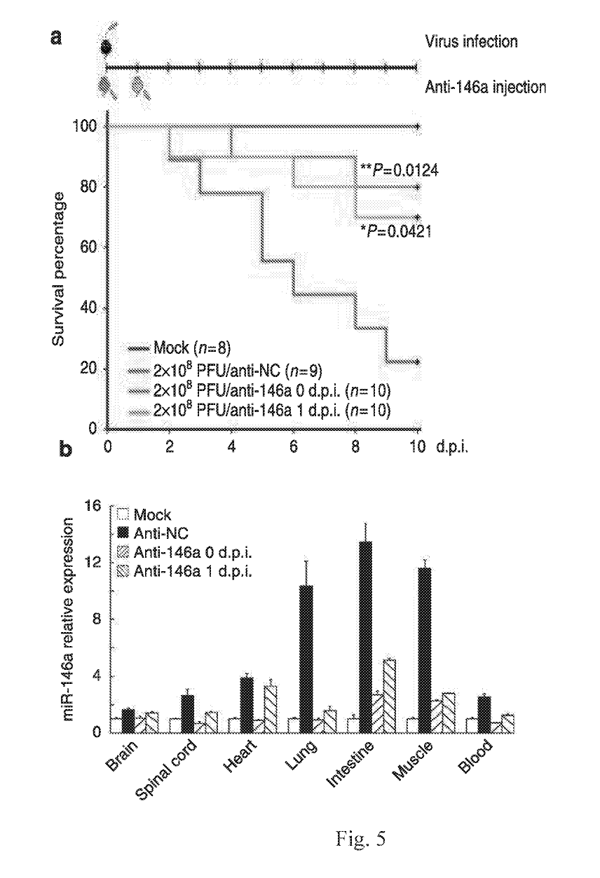

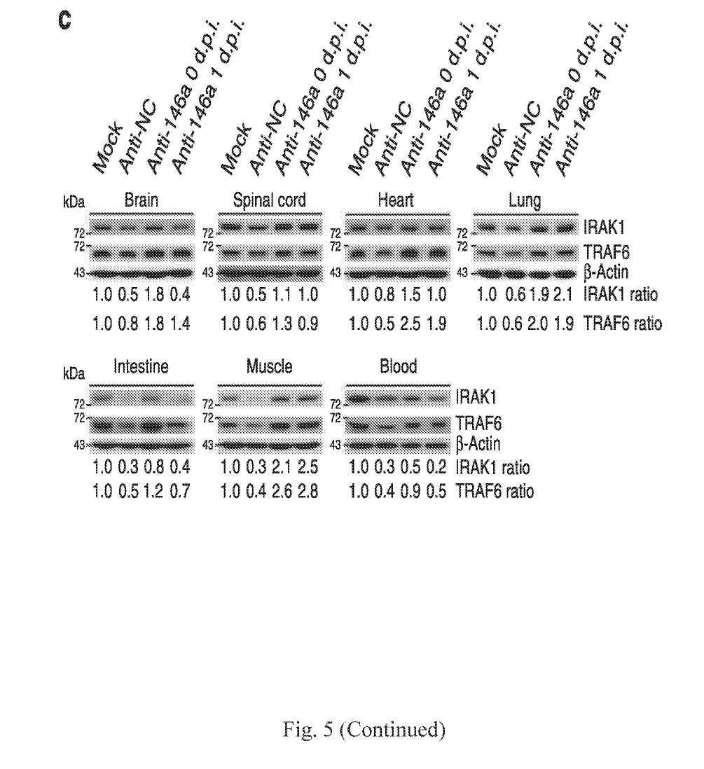

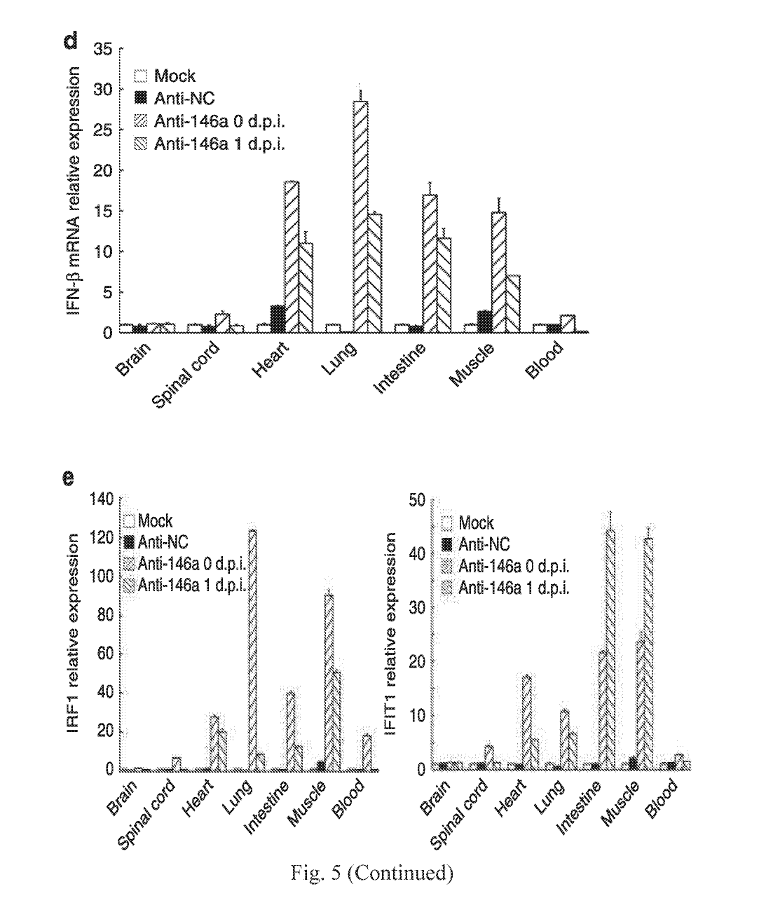

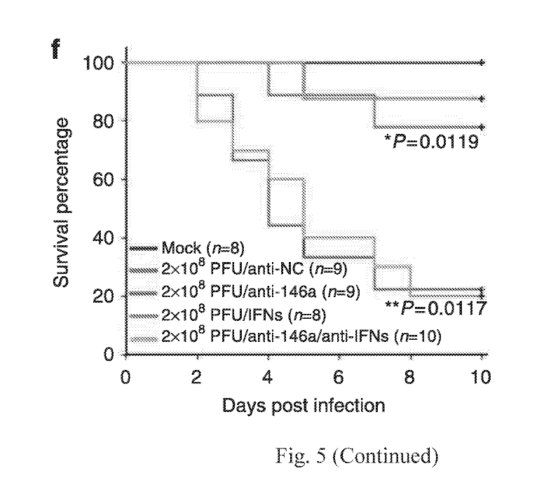

The sequence of LNA antagomiR-146a used in the example is 5′-AACCCATGGAATTCAGTTCTCA-3′ (SEQ ID NO:20). Even though the importance of miR-146a in EV71 pathogenesis was clearly elucidated by using miR-146a^{-/-} mice model, however, the therapeutic potential of miR-146a silencing should be practically evaluated in EV71 infection mouse model. LNA antagomiR-146a, designed locked nucleic acid, was first injected intraperitoneally to evaluate the potential adverse events caused by LNA antagomiRs. The HE staining and blood chemistry report obtained from liver, kidney and serum showed no significant pathological changes after LNA antagomiR-NC or LNA antagomiR-146a infection. After making sure the safety of LNA antagomiRs, we next introduced LNA AntagomiR-146a into wild-type mice before or after virus infection as indicated and monitored clinical symptoms of mice daily. Injection of LNA antagomiR-146a at 1 hour before virus infection (designated as 0 d.p.i.), 1 d.p.i. and 2 d.p.i. showed obvious improvement in survival (80%, 70% and 56%, respectively) compared with PBS or LNA antagomiR negative control group (22% and 25%, respectively) (FIG. 5a). Nevertheless, injection of LNA antagamiR-146a at 3 d.p.i. did not improve mice survival, and these data implied LNA antagomiR-146a might be used as the preventive agent as well as the therapeutic agent in the early phase of mEV71 infection. The expressions of miR-146a and two miR-146a targets, IRAK1 and TRAF6, were measured by real-time RT-PCR and Western blot, respectively, miR-146a was upregulated in mEV71-infected mice treated with LNA antagomiR negative control compared to mock infection. The virus-induced miR-146a was dramatically suppressed by LNA antagomiR-146a treatment at 0 and 1 d.p.i. particularly in the highly susceptible organs including intestine, muscle and lung (FIG. 5b). To verify whether attenuation of virus-induced miR-146a by LNA antagomiR-146a could restore the expression of miR-146a targets, IRAK1 and TRAF6 in assayed organs were measured by Western blot. FIG. 5c showed that IRAK1 and TRAF6 were restored by LNA antagomiR-146a specifically in heart, lung, and muscle and the results showed reverse correlation with suppression of miR-146a (FIGS. 5b-5c). In agreement with the findings in miR-146a^{-/-} mice study, regain of IFNβ production was found in EV71-infected wild-type mice treated with LNA antagomiR-146a at 0 and 1 d.p.i. compared with mock infection and LNA antagomiR negative control groups (FIG. 5d).

To determine whether IFNs indeed played critical roles in antagomiR-146a-mediated improved survival, we injected mice with LNA antagomiR-146a and anti-IFNα/β antibodies sequentially before (0 d.p.i.) virus infection and recorded mice survival daily. FIG. 5e showed anti-IFNα/β antibodies eliminated antagomiR-146a-mediated improved survival and meant that LNA antagomiR-1146a improved mice survival through regain of IFNs. Consequently, the improved survival probabilities observed in 0 and 1 d.p.i. groups were attributed to attenuation of virus-induced miR-146a, restoration of IRAK1 and TRAF6, and regain of IFNβ expression.

### Example 5

### Effects of Designed AntagomiR-146a1 and AntagomiR-146a2

The sequences of AntagomiR-146a1 and AntagomiR-146a2 are 5′-ATGGAATTCAGTTCTCA-3′ (SEQ ID NO:21) and 5′-ATTCAGTTCTCA-3′ (SEQ ID NO:22), respectively.

Cell Cultures and Virus Infection. Human rhabdomyosarcoma cells line (RD) and colon adenocarcinoma cell line (Caco-2) were cultured in MEM medium with 1 mM L-glutamine, 100 units/ml penicillin, and 100 μg/ml streptomycin (Life Technologies). Mediums for RD and Caco-2 cells were supplemented with 10% and 20% fetal bovine serum, respectively (Life Technologies). THP-1 cells, a kind of human monocytic cells derived from an acute monocytic leukemia patient, were cultured in RPMI-1640 medium with 5 mM L-glutamine, 100 units/ml penicillin, 100 μg/ml streptomycin and 10% fetal bovine serum. THP-1 cells were treated with PMA (phorbol-12-myristate-13-acetate) and differentiated into monocyte-derived macrophages. RD cells were used in propagation and plaque titration of poliovirus type 3 (PV3, Sabin strain), coxsackievirus B3 (CVB3), and enterovirus 71 (EV71). The virus infection was performed in the serum-free condition. Aliquots of viral stocks were stored at −80° C. All cell lines were obtained from ATCC source.

RNA Extraction and miRNA Profiling. RNAs were extracted from virus-infected or mock-infected RD cells by Trizol reagent (Life Technologies). The expression levels of 250 human miRNAs were measured using the TaqMan MicroRNA Assays (Life Technologies).

Individual Real-Time RT-PCR. Quantification of miR-146a, Homo RNU6B, mus U6 snRNA , mus IFNβ, and mus β-actin were performed using TaqMan microRNA individual assays or TaqMan gene expression assays (000468, 001093, 001973, Mm00439546_s1 and Mm00607939_s1; Life Technologies) according to the manufacturer's instructions. In brief, real-time RT-PCR was performed using a standard protocol on an Applied Biosystems 7900HT System. The 10 μl PCR mixture included 2 μl RT product, 5 μl 2× TaqMan Universal PCR Master Mix, 0.5 μl 20× TaqMan probe and primers, and 2.5 μl H₂O. The reactions were incubated in a 96-well plate at 95° C. for 10 min, followed by 40 cycles of 95° C. for 15 s and 60° C. for 1 min. All reactions were run in triplicate. The threshold cycle (CT) is defined as the fractional cycle number at which the fluorescence passes the fixed threshold. Quantification of c-jun, c-fos and TBP were performed by SYBR Green-based real-time PCR (Table 1).

Western Blot. Cells or tissues were harvested in RIPA lysis buffer [50 mM Tris-HCl (pH 7.4), 150 mM NaCl, 1 mM EDTA, 1% Triton X-100, 0.1% SDS, 1% sodium deoxycholate, 1 mM PMSF, and protease inhibitor cocktail], and the protein concentration was measured by the BCA protein assay (BioRad). Proteins were resolved by 10% sodium dodecyl sulfate polyacryhuide gel electrophoresis, transferred onto PVDF membranes, blocked with 5% skimmed milk in Tris-buffered saline (TBS) [20 mM Tris-HCl (pH 7.5), 150 mM NaCl, and 0.5% Tween-20] and reacted with primary antibodies for β-actin (1:5000; Sigma), Homo TRAF6 (1:200; Santa Cruz), Homo IRAK1 (1:200; Santa Cruz), c-jun (1:200; Santa Cruz), c-fos (1:200; Santa Cruz), Re1A (1:500; Biolegend), Histone H3 (1:3000; Cell Signaling), mus TRAF6 (1:200; Santa Cruz), mus IRAK1 (1:200; Santa Cruz), and V5 tag (1:5000; Life Technologies). β-actin acted as an internal control.

Luciferase Assay. All transfections were carried out in triplicate in 96-well plates. RD cells (1×10⁴ per well) were seeded 24 h prior to transfection. The luciferase reporter constructs along with the control plasmids (pRL-TK Vector; Promega) were co-transfected into cells at the DNA ratio 5:1 in the presence of pSilencer miRNA expressing vectors (Life Technologies) as indicated by Lipofectamine LTX reagent (Life Technologies). After 48 h incubation, the Dual-Glo luciferase substrate (Promega) was added to each well and the luminescent signals were measured by Victor3 multilabel counter (PerkinElmer) according to the manufacturer's instructions. For IFNβ promoter assays, the reporter constructs were co-transfected with antagomiR-NC or antagomiR-146a into RD cells prior to virus infection. After 24 h incubation, all transfectants were infected with EV71 and assayed at indicated time points. The activity of Renilla luciferase was used as an internal control to normalize transfection efficiency.

Plasmid Constructions. The full-length TRAF6 and IRAK1 3′UTR were amplified from complementary DNAs of RD cells by using TRAF6 luc F/TRAF6 luc R and IRAK1 luc F/IRAK1 luc R, respectively (Table 1). Paired primers (TRAF6 luc F and TRAF6 mut R1, TRAF6 mut F1 and TRAF6 luc R, TRAF6 luc F and TRAF6 mut R2, TRAF6 mut F2 and TRAF6 luc R, TRAF6 luc F and TRAF6 mut R3, and TRAF6 mut F3 and TRAF6 luc R) were used to generate the mutant-types of TRAF6 3′UTR, in which the four mutated nucleotides were underlined within the seed region of miR-146a binding site by PCR-based mutagenesis method (Table). For mutant-types of IRAK1 3′UTR constructs, primers were designated as IRAK1 luc F, IRAK1 luc R, IRAK1 mut F1, IRAK1 mut R1, IRAK1 mut F2, and IRAK1 mut R2 (Table 1). All PCR fragments were cloned into pMIR-reporter luciferase vector (Life Technologies). The coding regions and 3′UTRs of TRAF6 and IRAK1 fragments were amplified from cDNAs of RD cells and cloned into pcDNA 3.1 expression vector (Life Technologies) along with V5 tag. The miR-146a precursor fragment was amplified by PCR-based ligation and constructed into pSilencer vector (Life Technologies) with BamHI and HindIII (Table 1). The promoter regions of miR-146a precursor and IFNβ were constructed into pGL3 basic vectors, respectively.

TABLE 1
Primer list

Primers Sequence (5′ to 3′)

Luciferase Reporter Vector

| | | |
|---|---|---|
| IRAK1 luc F | 5′-actagtATGTGTTCACCTGGGCAGATC-3′ | (SEQ ID NO. 23) |
| IRAK1 luc R | 5′-gtttaaacTTATTGCAACATACGTTTTTATTAC-3′ | (SEQ ID NO. 24) |
| IRAK1 mut F1 | 5′-GAAGTCAAAGTAGAGTTGGTCAGAAG-3′ | (SEQ ID NO. 25) |
| IRAK1 mut R1 | 5′-CTTCTGACCAACTCTACTTTGACT-3′ | (SEQ ID NO. 26) |
| IRAK1 mut F2 | 5′-TGGTGAGAAGTAGAGTTGGTGCACGA-3′ | (SEQ ID NO. 27) |
| IRAK1 mut R2 | 5′-TCGTGCACCAACTCTACTTCTGACCA-3′ | (SEQ ID NO. 28) |
| TRAF6 luc F | 5′-gagctcGCTTGCCCTCACTTGCTCA-3′ | (SEQ ID NO. 29) |
| TRAF6 luc R | 5′-gccggcTTAACACTTAAACAAGTATTATTCAA-3′ | (SEQ ID NO. 30) |
| TRAF6 mut F1 | 5′-TGCCCTGTAGAGTATAACAT-3′ | (SEQ ID NO. 31) |
| TRAF6 mut R1 | 5′-ATGTTATACTCTACAGGGCA-3′ | (SEQ ID NO. 32) |
| TRAF6 mut F2 | 5′-AAGTTGAGTAGAGTTTTTTTTA-3′ | (SEQ ID NO. 33) |
| TRAF6 mut R2 | 5′-TAAAAAAAACTCTACTCAACTT-3′ | (SEQ ID NO. 34) |
| TRAF6 mut F3 | 5′-ACTTAAGTAGAGTTTCACCC-3′ | (SEQ ID NO. 35) |
| TRAF6 mut R3 | 5′-GGGTGAAACTCTACTTAAGT-3′ | (SEQ ID NO. 36) |

Ectopic Expression Vector

| | | |
|---|---|---|
| c-jun F | 5′-aagcttATGACTGCAAAGATGGAAAC-3′ | (SEQ ID NO. 37) |
| c-jun R | 5′-ctcgagTCAAAATGTTTGCAACTGCT-3′ | (SEQ ID NO. 38) |
| c-fos F | 5′-aagcttATGATGTTCTCGGGCTTCAA-3′ | (SEQ ID NO. 39) |
| c-fos R | 5′-ctcgagTCACAGGGCCAGCAGCG-3′ | (SEQ ID NO. 40) |

SYBR Green Assay

| | | |
|---|---|---|
| c-jun SYBR F | 5′-ACCGCTGCGCACGAA-3′ | (SEQ ID NO. 41) |
| c-jun SYBR R | 5′-GCTACCCGGCTTTGAAAAGTC-3′ | (SEQ ID NO. 42) |
| c-fos SYBR F | 5′-ATGGGCTCGCCTGTCAAC-3′ | (SEQ ID NO. 43) |
| c-fos SYBR R | 5′-CAGTGACCGTGGGAATGAAGT-3′ | (SEQ ID NO. 44) |
| IRAK1 SYBR F | 5′-GGGCGGTGATGAGGAACA-3′ | (SEQ ID NO. 45) |
| IRAK1 SYBR R | 5′-CCACTCCAGGTCAGCGTTCT-3′ | (SEQ ID NO. 46) |
| TRAF6 SYBR F | 5′-CCATGCGGCCATAGGTTCT-3′ | (SEQ ID NO. 47) |
| TRAF6 SYBR R | 5′-TTGTGACCTGCATCCCTTATTG-3′ | (SEQ ID NO. 48) |
| TBP SYBR F | 5′-CACGAACCACGGCACTGATT-3′ | (SEQ ID NO. 49) |
| TBP SYBR R | 5′-TTTTCTTGCTGCCAGTCTGGAC-3′ | (SEQ ID NO. 50) |

pSilencer Vector

| | | |
|---|---|---|
| miR-146a 1F | 5′-GATCCCCGATGTGTATCCTCAGCTTTGAGAACT-3′ | (SEQ ID NO. 51) |
| miR-146a 1R | 5′-GGAATTCAGTTCTCAAAGCTGAGGATACACATCGGG-3′ | (SEQ ID NO. 52) |
| miR-146a 2F | 5′-GAATTCCATGGGTTGTGTCAGTGTCAGACCTCTGA-3′ | (SEQ ID NO. 53) |
| miR-146a 2R | 5′-CTGAATTTCAGAGGTCTGACACTGACACAACCCAT-3′ | (SEQ ID NO. 54) |
| miR-146a 3R | 5′-AATTCAGTTCTTCAGCTGGGATATCTCTGTCATCGTA-3′ | (SEQ ID NO. 55) |
| miR-146a 3R | 5′-AAGCTACGATGACAGAGATATCCCAGCTGAAGAA-3′ | (SEQ ID NO. 56) |

Stable Transfection of RD Cells and AntagomiR Transfections. To generate the stably TRAF6- or IRAK1-expressing cell lines, RD cells were transfected with 2 μg of plasmid DNA encoding V5-TRAF6 or V5-IRAK1 fusion protein with wild-type or mutant 3′UTR by using Lipofectamine LTX reagent (Life Technologies) and treated with G418 (1 mg/ml; Life Technologies). For antagomiR transfection, trypsinized RD cells at 3×10⁵/ml were transfected with control antagomiR (5 pM) or specific antagomiR (5 pM) (Life Technologies) by siPORT NeoFX transfection reagent (Life Technologies) according to the manufacturer's instructions.

Plaque Assay. EV71 plaque assays were carried out in triplicate in 6-well plates. RD cells were infected with 100 μl per well of diluted viral stocks. After 1 h incubation, the infected cells were washed and incubated for 3 days in 0.3% agarose medium overlay. Cells were fixed with formaldehyde and stained with crystal violet. Plaques were counted.

JNK Inhibitor Treatment. RD cells were seeded into 6-well plates and infected with EV71 under 20 μM JNK inhibitor (SP600125, Sigma-Aldrich), DMSO, or medium only. After infection, total proteins and RNAs were extracted and assayed with Western blot or real-time RT-PCR, respectively.

Mouse-adapted EV71 and LNA AntagomiRs Administration. C57BL/6 mice were provided by the Knockout Mouse Core Laboratory of National Taiwan University Center of Genomic Medicine, housed in specific pathogen-free animal rooms, and treated according to guidelines from the National Taiwan University College of Medicine and College of Public Health Institutional Animal Care and Use Committee (IACUC). Mouse-adapted EV71 (mEV71) was established referring to a report published by Wang, Y. F. in 2004. mEV71 was generated after four serial passages in neonate mice started from parental human EV71 Parental human EV71 was injected intraperitoneally and next generation mEV71, called 1st mEV71, was isolated from neonate mice brain tissue at 3 d.p.i. The isolated 1st mEV71 was then propagated in RD cells. The passage procedures were performed four times. To determine the 50% lethal dose (LD50), seven-day-old wild-type C57BL/6 mice were fed with indicated PFUs of mEV71. The survival of mice was monitored daily. For wild-type C57BL/6 mice and miR-146a−/− C57BL/6 mice inoculation, each group (n=9 to 13) housed in the same cage was infected with indicated PFUs of mEV71 through the oral route, and the control group was fed with culture medium. The animals were monitored daily, and clinical signs, weight and mortality were recorded. All mouse tissues were obtained from scarified mice with significant clinical illness signs or at 3 d.p.i. if mice had no significant illness signs. The tissues were further assayed for real-time RT-PCR, Western blot, plaque assay, immunohistochemistry staining, and so on. For LNA antagomiRs injection, wild-type mice were injected with LNA antagomiR-146a (1.2 mg/kg) or LNA antagomiR negative control (1.2 mg/kg) through the intraperitoneal route before or after virus infection as indicated and monitored daily. The institutional animal care and use committee (IACUC) approved all animal protocols.

Immunohistochemistry Staining. Mock-infected and virus-infected mouse sections for immunostaining were obtained from optimal cutting temperature (OCT)-embedded tissues. The samples were stained with primary antibodies anti-Enterovirus 71 (1:200; Millipore) at 4° C. for 12 h. The samples were washed twice with PBS, treated with goat anti-mouse IgG biotin-labeled secondary antibody (1:500; Vector Laboratories) at room temperature for 1 h, and developed by ABC kit (Vector Laboratories) according to the manufacturer's instructions. The slides were then examined by microscope.

Statistical Analysis. Student's t test was used to compare the miRNA expression at different time points during EV71 infection. p value <0.05 was considered as significant and two-tailed tests were used in this study. The miRNAs with greater than 2-fold change of expressions at both 4 and 8 h.p.i. relative to mock infection were identified for further study. The associations of miR-146a with IRAK1 or TRAF6 and IRAK1 or TRAF6 with IFNβ are presented as coefficient of correlation (r) estimated by Pearson correlation method. The positive or negative r represents the positive or negative association between the two variables.

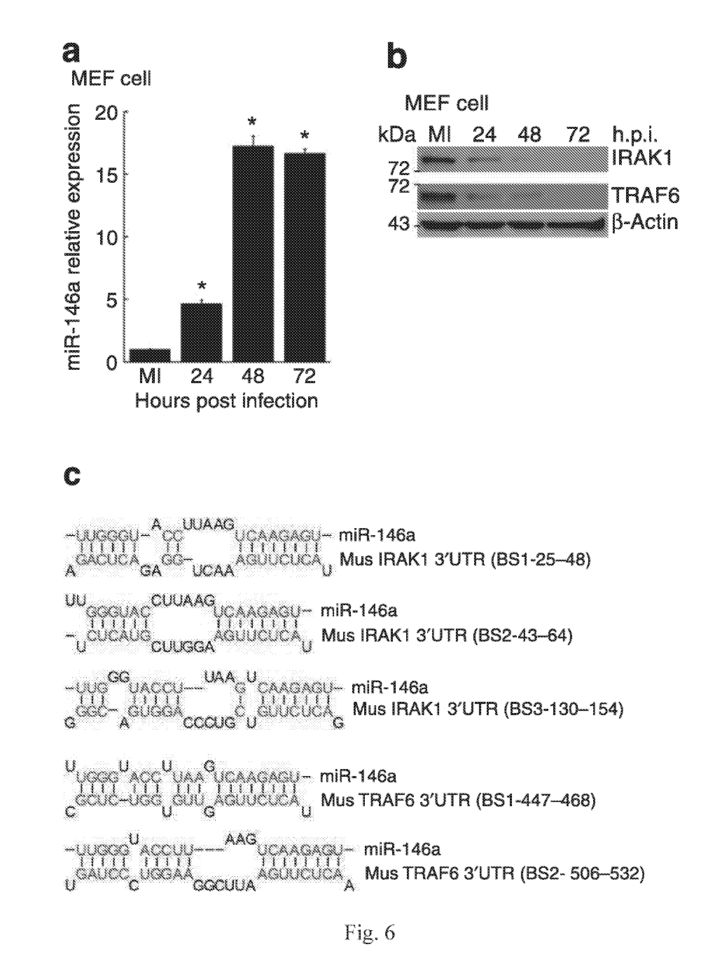

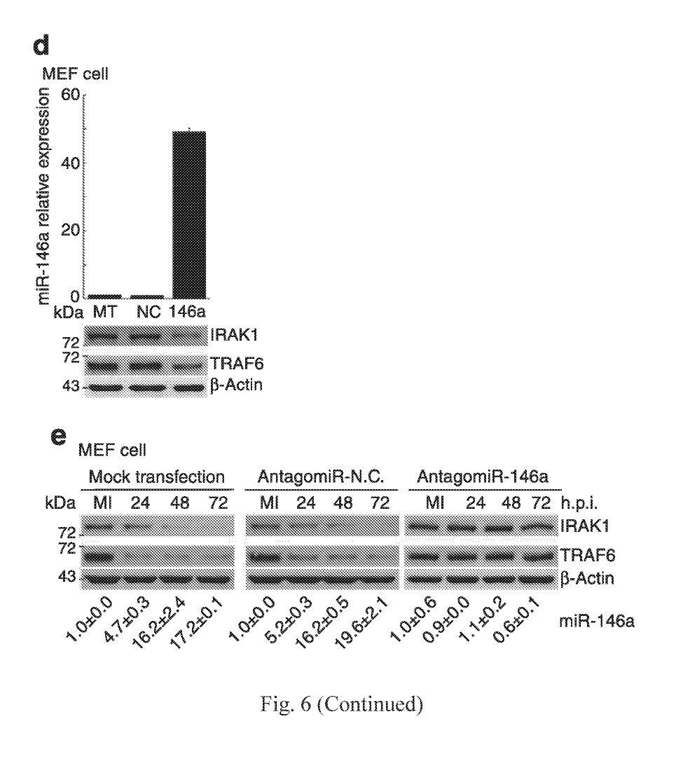

As shown in FIG. 6, (a) miR-146a was induced in mEV71 infection. MEF cells were infected with mEV71, followed by quantification of miR-146a using real-time RT-PCR (n=3). MI, mock infection. * P-value <0.05 as compared with mock infection. (b) mEV71 infection suppressed IRAK1 and TRAF6 expressions in protein level in MEF cells. (c) Predicted mus miR-146a BSs within mus IRAK1 and TRAF6 3′UTRs (the first nucleotide following the stop codon was designated as +1). (d) The effect of the ectopic miR-146a on the endogenous IRAK1 and TRAF6. MEF cells were transfected with the miR-146a overexpressing vector or negative control and assayed for IRAK1 and TRAF6 and for miR-146a by western blot and real-time RT-PCR, respectively (n=3). MT, mock transfection; NC, negative control. (e) AntagomiR-146a restored mEV71-induced suppression of TRAF6 and IRAK1 in MEF cells. MEF cells were transfected with antagomiR-146a or antagomiR-NC followed by mEV71 infection. All data presented are mean±s.d. and all P-values are calculated by Student's t-test.

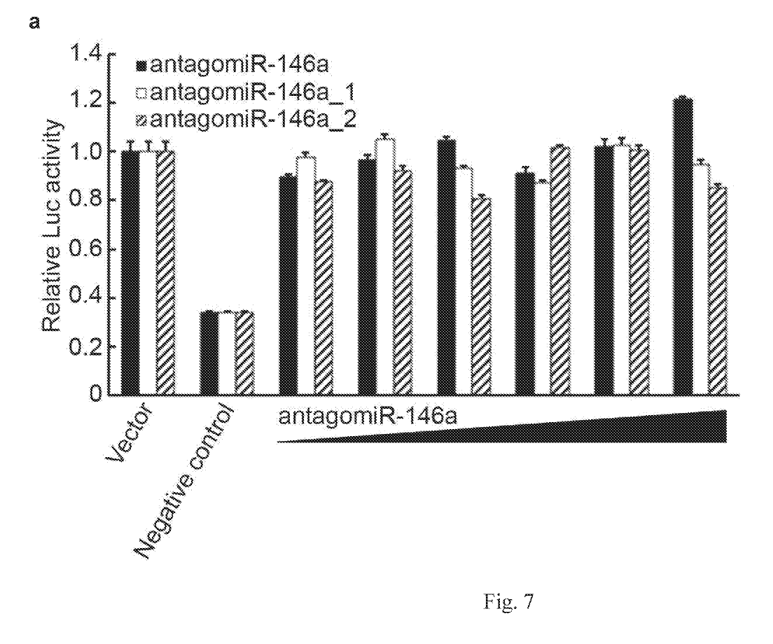

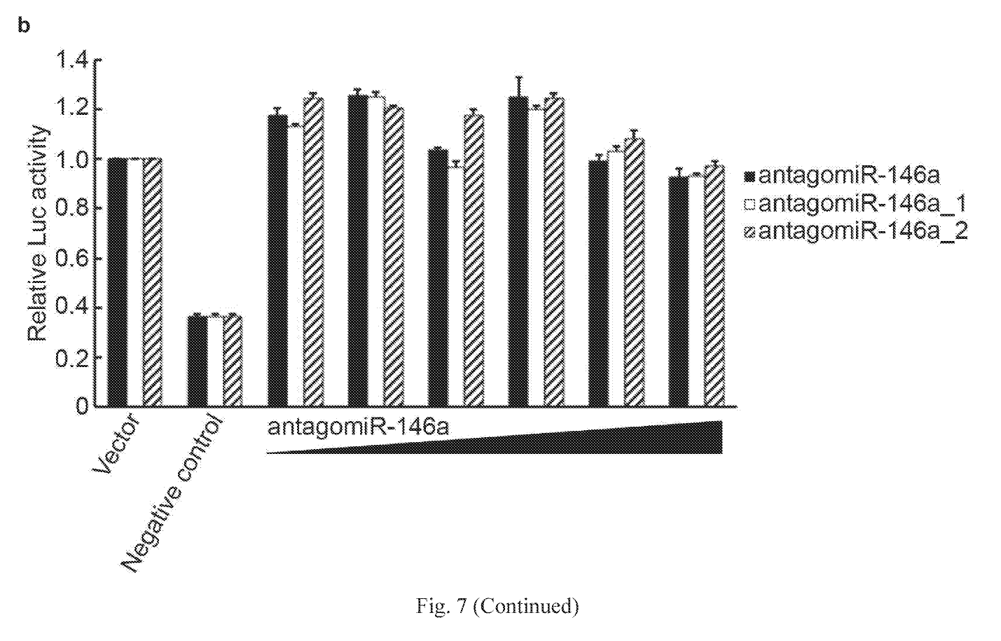

FIG. 7 shows that the indicated concentrations of designed antagomiR-146a were transfected into RD cells in the presence of pSilencer-miR-146a. The inhibitory activities of pSilencer-146a on pMIR-IRAK1 3′UTR (A) and pMIR-TRAF6 3′UTR (B) were restored by antagomiR-146a introductions compared with antagomiR-NC.

What is claimed is:

1. A single strand oligonucleotide or a nucleotide analogue thereof, which has a length of 8-25 nucleobase units, wherein the oligonucleotide comprises a seed nucleobase sequence consisting of AGTTCTCA (SEQ ID NO: 1) counting from 3′ end of the oligonucleotide.

2. The single strand oligonucleotide or a nucleotide analogue thereof of claim 1, wherein at least about 50% of the nucleobase units of the single stranded oligonucleotide are complementary to the miR-146a sequence or a region thereof.

3. The single strand oligonucleotide or a nucleotide analogue thereof of claim 1, wherein at least about 95% of the nucleobase units of the single stranded oligonucleotide are complementary to the miR-146a sequence or a region thereof.

4. The single strand oligonucleotide or a nucleotide analogue thereof of claim 1, which comprises a contiguous nucleotide sequence fully complementary to the sequence of the seed region of miR-146a.

5. The single strand oligonucleotide or a nucleotide analogue thereof of claim 1, which comprises a contiguous nucleotide sequence which is fully complementary to at least 12 contiguous nucleotides present in the sequence of miR-146a.

6. The single strand oligonucleotide or a nucleotide analogue thereof of claim 1, which comprises a contiguous nucleotide sequence which is fully complementary to at least 17 contiguous nucleotides present in the sequence of miR-146a.

7. The single strand oligonucleotide or a nucleotide analogue thereof according to claim 5, wherein the contiguous nucleotide sequence of the oligomer is fully complementary to the sequence of a region of miR-146a.

8. The single strand oligonucleotide or a nucleotide analogue thereof according to claim 1, wherein the contiguous nucleotide sequence of the oligomer comprises between 12 and 22 nucleotides which are fully complementary to a sequence of miR-146a.

9. The single strand oligonucleotide or a nucleotide analogue thereof according to claim 1, which comprises one or more 2′-O-methoxyethyl-RNA, 2′-fluoro-DNA monomers or LNA unit.

10. The single strand oligonucleotide or a nucleotide analogue thereof according to claim 1, which is selected from the group consisting of:
5′-AACCCATGGAATTCAGTTCTCA-3′;
5′-AACCCB(T, G, C)TGGAATTCAGTTCTCA-3′;
5′-AACCD(T, A, G)ATGGAATTCAGTTCTCA-3′;
5′-AAD(T, A, G)CCATGGAATTCAGTTCTCA-3′;
5′-AB(T, G, C)CCCATGGAATTCAGTTCTCA-3′;
5′-B(T, G, C)ACCCATGGAATTCAGTTCTCA-3′;
5′-N(additional A, T, C, G)
AACCCATGGAATTCAGTTCTCA-3′;
5′-NN(additional A, T, C, G)
AACCCATGGAATTCAGTTCTCA-3′;
5′-NNN(additional A, T, C, G)
AACCCATGGAATTCAGTTCTCA-3′;
5′-AACCCATGGAATTCAGTTCTCA-3′;
5′-ATGGAATTCAGTTCTCA-3′;
and
5′-ATTCAGTTCTCA-3′

11. A pharmaceutical composition, comprising the single strand oligonucleotide or a nucleotide analogue thereof according to claim 1.

12. A method for diagnosis of the infection caused by Picornavirus, comprising the steps of:
(a) providing a sample of a subject supposed to suffer from Picornavirus infection;
(b) measuring the expression of miR-146a, c-jun, c-fos, IRAK1 and/or TRAF6;
wherein an elevated level of miR-146a and an elevated level of c-jun and/or c-fos or a reduced level of IRAK1 and/or TRAF6 in comparison to a control sample indicates Picornavirus infection.

13. A method for screening of a pharmaceutically active compound for the treatment and/or the prevention of Picornavirus infection, comprising the steps of:
(a) providing a cell infected with Picornavirus;
(b) contacting a candidate substance with the cell; and
(c) measuring the expression or promoter activity of miR-146a, c-jun, c-fos, IRAK1 and/or TRAF6 in the cell;
wherein a reduced level of miR-146a and a reduced level of c-jun and/or c-fos or an elevated level of IRAK1 and/or TRAF6 in comparison to a control sample indicates a pharmaceutically active compound.

14. The method of claim 12 or 13, wherein miR-146a, c-jun, c-fos, IRAK1 and TRAF6 are measured.

15. A method for neutralizing Picornavirus, comprising contacting a miR-146a antagnoist with the Picornavirus, wherein the miR-146a antagonist is the single strand oligonucleotide or a nucleotide analogue thereof of claim 1.

16. A method for treating and/or preventing Picornavirus infection, comprising administering an effective amount of miR-146a antagnoist to a subject, wherein the miR-146a antagonist is the single strand oligonucleotide or a nucleotide analogue thereof of claim 1.

17. The method of claim 15 or 16, wherein the miR-146a antagnoist is any one of the single strand oligonucleotide or a nucleotide analogue thereof of claim 10.

18. The method according to any of claims 12, 13, 14 and 15, wherein the Picornavirus is Enterovirus.

19. The method according to any of claims 18, wherein the Enterovirus is Enterovirus A, Enterovirus B or Enterovirus C.

20. The method according to any of claims 18, wherein the Enterovirus is Enterovirus 71.


SEQUENCE LISTING (56 sequences)

SEQ ID NO: 1 - DNA, 22 bp, Artificial Sequence
  miR-146a sequence
uuggguaccuuaagucaagagu

SEQ ID NO: 2 - DNA, 22 bp, Artificial Sequence
  single stranded oligonucleotide
ugagaacugaauuccauggguu

SEQ ID NO: 3 - DNA, 22 bp, Artificial Sequence
  antimiR-146a sequence
aacccatggaattcagttctca

SEQ ID NO: 4 - DNA, 22 bp, Artificial Sequence
  single stranded oligonucleotide
ugagaacugaauuccavggguu

SEQ ID NO: 5 - DNA, 22 bp, Artificial Sequence
  antimiR-146a sequence
aacccbtggaattcagttctca

SEQ ID NO: 6 - DNA, 22 bp, Artificial Sequence
  single stranded oligonucleotide
ugagaacugaauuccauhgguu

SEQ ID NO: 7 - DNA, 22 bp, Artificial Sequence
  antimiR-146a sequence
aaccdatggaattcagttctca

SEQ ID NO: 8 - DNA, 22 bp, Artificial Sequence
  single stranded oligonucleotide
ugagaacugaauuccaugghuu

SEQ ID NO: 9 - DNA, 22 bp, Artificial Sequence
  antimiR-146a sequence
aadccatggaattcagttctca

SEQ ID NO: 10 - DNA, 22 bp, Artificial Sequence
  single stranded oligonucleotide
ugagaacugaauuccaugggvu

SEQ ID NO: 11 - DNA, 22 bp, Artificial Sequence
  antimiR-146a sequence
abcccatggaattcagttctca

SEQ ID NO: 12 - DNA, 22 bp, Artificial Sequence
  single stranded oligonucleotide
ugagaacugaauuccauggguv

SEQ ID NO: 13 - DNA, 22 bp, Artificial Sequence
  antimiR-146a sequence
bacccatggaattcagttctca

SEQ ID NO: 14 - DNA, 23 bp, Artificial Sequence
  single stranded oligonucleotide
ugagaacugaauuccauggguun

SEQ ID NO: 15 - DNA, 23 bp, Artificial Sequence
  antimiR-146a sequence
naacccatggaattcagttctca

SEQ ID NO: 16 - DNA, 24 bp, Artificial Sequence
  single stranded oligonucleotide
ugagaacugaauuccauggguunn

SEQ ID NO: 17 - DNA, 24 bp, Artificial Sequence
  antimiR-146a sequence
nnaacccatggaattcagttctca

SEQ ID NO: 18 - DNA, 25 bp, Artificial Sequence
  single strand oligonucleotide
ugagaacugaauuccauggguunnn

SEQ ID NO: 19 - DNA, 25 bp, Artificial Sequence
  antimiR-146a sequence
nnnaacccatggaattcagttctca

SEQ ID NO: 20 - DNA, 22 bp, Artificial Sequence
  single stranded oligonucleotide
aacccatggaattcagttctca

SEQ ID NO: 21 - DNA, 17 bp, Artificial Sequence
  single strand oligonucleotide
atggaattcagttctca

SEQ ID NO: 22 - DNA, 12 bp, Artificial Sequence
  single stranded oligonucleotide
attcagttctca

SEQ ID NO: 23 - DNA, 27 bp, Artificial Sequence
  primer
actagtatgtgttcacctgggcagatc

SEQ ID NO: 24 - DNA, 33 bp, Artificial Sequence
  primer
gtttaaacttattgcaacatacgtttttattac

SEQ ID NO: 25 - DNA, 26 bp, Artificial Sequence
  primer
gaagtcaaagtagagttggtcagaag

SEQ ID NO: 26 - DNA, 24 bp, Artificial Sequence
  primer
cttctgaccaactctactttgact

SEQ ID NO: 27 - DNA, 26 bp, Artificial Sequence
  primer
tggtcagaagtagagttggtgcacga

SEQ ID NO: 28 - DNA, 26 bp, Artificial Sequence
  primer
tcgtgcaccaactctacttctgacca

SEQ ID NO: 29 - DNA, 25 bp, Artificial Sequence
  primer
gagctcgcttgccctcacttgctca

SEQ ID NO: 30 - DNA, 32 bp, Artificial Sequence
  primer
gccggcttaacacttaaacaagtattattcaa

SEQ ID NO: 31 - DNA, 20 bp, Artificial Sequence
  primer
tgccctgtagagtataacat

SEQ ID NO: 32 - DNA, 20 bp, Artificial Sequence
  primer
atgttatactctacagggca

SEQ ID NO: 33 - DNA, 22 bp, Artificial Sequence
  primer
aagttgagtagagtttttttta

SEQ ID NO: 34 - DNA, 22 bp, Artificial Sequence
  primer
taaaaaaaactctactcaactt

SEQ ID NO: 35 - DNA, 20 bp, Artificial Sequence
  primer
acttaagtagagtttcaccc

SEQ ID NO: 36 - DNA, 20 bp, Artificial Sequence
  primer
gggtgaaactctacttaagt

SEQ ID NO: 37 - DNA, 26 bp, Artificial Sequence
  primer
aagcttatgactgcaaagatggaaac

SEQ ID NO: 38 - DNA, 26 bp, Artificial Sequence
  primer
ctcgagtcaaaatgtttgcaactgct

SEQ ID NO: 39 - DNA, 26 bp, Artificial Sequence
  primer
aagcttatgatgttctcgggcttcaa

SEQ ID NO: 40 - DNA, 23 bp, Artificial Sequence
  primer
ctcgagtcacagggccagcagcg

SEQ ID NO: 41 - DNA, 15 bp, Artificial Sequence
  primer
accgctgcgcacgaa

SEQ ID NO: 42 - DNA, 21 bp, Artificial Sequence
  primer
gctacccggctttgaaaagtc

SEQ ID NO: 43 - DNA, 18 bp, Artificial Sequence
  primer
atgggctcgcctgtcaac

SEQ ID NO: 44 - DNA, 21 bp, Artificial Sequence
  primer
cagtgaccgtgggaatgaagt

SEQ ID NO: 45 - DNA, 18 bp, Artificial Sequence
  primer
gggcggtgatgaggaaca

SEQ ID NO: 46 - DNA, 20 bp, Artificial Sequence
  primer
ccactccaggtcagcgttct

SEQ ID NO: 47 - DNA, 19 bp, Artificial Sequence
  primer
ccatgcggccataggttct

SEQ ID NO: 48 - DNA, 22 bp, Artificial Sequence
  primer
ttgtgacctgcatcccttattg

SEQ ID NO: 49 - DNA, 20 bp, Artificial Sequence
  primer
cacgaaccacggcactgatt

SEQ ID NO: 50 - DNA, 22 bp, Artificial Sequence
  primer
ttttcttgctgccagtctggac

SEQ ID NO: 51 - DNA, 33 bp, Artificial Sequence
  primer
gatccccgatgtgtatcctcagctttgagaact

SEQ ID NO: 52 - DNA, 36 bp, Artificial Sequence
  primer
ggaattcagttctcaaagctgaggatacacatcggg

SEQ ID NO: 53 - DNA, 35 bp, Artificial Sequence
  primer
gaattccatgggttgtgtcagtgtcagacctctga

SEQ ID NO: 54 - DNA, 35 bp, Artificial Sequence
  primer
ctgaatttcagaggtctgacactgacacaacccat

SEQ ID NO: 55 - DNA, 37 bp, Artificial Sequence
  primer
aattcagttcttcagctgggatatctctgtcatcgta

SEQ ID NO: 56 - DNA, 34 bp, Artificial Sequence
  primer
aagctacgatgacagagatatcccagctgaagaa